(19) United States
(12) Patent Application Publication
(10) Pub. No.: US 2016/0310567 A1
(43) Pub. Date: Oct. 27, 2016

(54) COMPOSITIONS FOR INHIBITING VIRAL REPLICATION AND METHODS OF USE AND PRODUCTION THEREOF

(71) Applicant: FLORIDA ATLANTIC UNIVERSITY, Boca Raton, FL (US)

(72) Inventor: Massimo Caputi, Delray Beach, FL (US)

(21) Appl. No.: 15/136,322

(22) Filed: Apr. 22, 2016

Related U.S. Application Data
(60) Provisional application No. 62/151,460, filed on Apr. 23, 2015.

(51) Int. Cl. A61K 38/17; C07K 14/47; A61K 9/127; A61K 47/48

(52) U.S. Cl. CPC A61K 38/1709; A61K 47/48315; C07K 14/4702; A61K 9/127; A61K 48/00

(57) ABSTRACT

Compositions for inhibiting replication of a virus in a subject (e.g., a human patient) infected with the virus or at risk of being infected with the virus include a therapeutically effective amount of SRSF1 protein, a portion of a SRSF1 protein, or a nucleic acid encoding the SRSF1 protein or a portion thereof, and a pharmaceutically acceptable carrier. Methods of using these compositions, and kits including these compositions, are also described herein. These compositions, kits and methods provide novel therapies for AIDS based on the delivery of SRSF1, single protein domains (portions) derived from SRSF1, and compounds mimicking the activity of such domains.

### CROSS-REFERENCE TO RELATED APPLICATIONS

This application claims the benefit of Provisional Application Ser. No. 62/151,460, filed Apr. 23, 2015, which is hereby incorporated by reference in its entirety, for all purposes, herein.

### FEDERALLY SPONSORED RESEARCH

This invention was made with government support under grant no. R15 AI093229 awarded by the National Institutes of Health. The U.S. government has certain rights in the invention.

### FIELD OF THE INVENTION

The invention relates generally to the fields of medicine, molecular biology, and virology. In particular, the invention relates to compositions, kits and methods for inhibiting viral replication, including replication of Human Immunodeficiency Virus (HIV).

### BACKGROUND

Despite considerable efforts, a vaccine or a cure for AIDS is still unavailable. The search for agents targeting individual stages in the HIV life cycle will therefore continue for the foreseeable future. The current treatment of HIV-1 infected patients is based on compounds that directly target the activity of proteins coded by the virus. However, viral proteins such as reverse transcriptase, protease and integrase mutate at a rapid rate, generating novel, drug resistant strains which appear with growing frequency in the infected population. Setbacks in the development of vaccines have further accentuated the need for novel drugs. Several cellular factors are required for the efficient expression of the HIV-1 genome. The identification and study of such factors might provide novel therapeutic targets, which offer clear advantages since they do not mutate in response to drugs and do not have a high degree of polymorphism in the population. Genomic screenings utilizing siRNA libraries helped in the identification of several cellular proteins required for efficient HIV-1 replication and infectivity. The factors isolated belong to a variety of functionally diverse protein families and a number of strategies aimed at limiting their activity in the viral life cycle are being developed. Nevertheless, technical and conceptual obstacles are hampering the development of novel therapies. Inhibition of the activity of cellular proteins by small chemical compounds may negatively affect cell viability and silencing the expression of cellular factors by antisense RNAs is often inefficient and can't target proteins with similar and redundant activities.

One shortcoming of genomic screenings based on the silencing of cellular genes is that they seldom identify factors that restrict viral expression. Screening complementary DNA libraries for clones able to induce resistance to infection has proved to be an excellent tool to identify new factors. However, although current approaches aimed at increasing the activity of viral restriction factors present some advantages, the expression or activation of cellular factors restricting viral growth is technically challenging. A need exists for novel therapeutics with diverse mechanisms of action.

### SUMMARY

Described herein are compositions, kits and methods for inhibiting replication of a virus in a subject (e.g., a subject infected by HIV) and preventing, ameliorating, or treating an HIV (e.g., HIV-1) infection in a subject. It was discovered that the cellular protein SRSF1 is a strong inhibitor of viral replication. Replication of the integrated HIV-1 genome is tightly regulated by a series of cellular factors. The experimental results described herein demonstrate that the cellular splicing factor SRSF1 can down-regulate replication of B, C and D subtype viruses by over 200 fold in a cell culture system. That viral transcription and splicing are inhibited by SRSF1 expression was also shown. Furthermore, SRSF1 deletion mutants containing the protein RNA binding domains (RBDs) but not the arginine serine rich (SR) domain can down-regulate viral replication by over 2,000 fold with minimal impact on cell viability and apoptosis. Given the strong antiviral properties of this protein and its RNA binding domains and the minimal effects observed on cell metabolism, these data suggest a therapeutic potential for SRSF1 and its RNA binding domains.

Accordingly, described herein is a composition including a therapeutically effective amount of SRSF1 protein, a portion of a SRSF1 protein, or a nucleic acid encoding the SRSF1 protein or a portion thereof, for inhibiting replication of a virus in a subject infected with the virus or at risk of being infected with the virus and a pharmaceutically acceptable carrier. The portion of a SRSF1 protein can include RNA Recognition Motif 1 (RRM-1) and RNA Recognition Motif 2 (RRM-2). The portion of a SRSF1 protein can include RRM-2. Typically, the virus is HIV-I and the subject is a human. The SRSF1 protein or the portion of a SRSF1 protein can be conjugated to a cell penetrating peptide (CPP). In one embodiment, the CPP is Tat-CPP. The composition can further include an endosomolytic agent (e.g., dfTat). The composition can include at least one of: an endosomolytic agent (e.g., dfTat) and a CPP. In some embodiments, a viral vector includes the nucleic acid encoding the SRSF1 protein or a portion thereof. Examples of viral vectors include: lentiviral vector, retroviral vector, Adeno-Associated Virus vector, Adenovirus vector, and Herpesvirus vector. In other embodiments, a cationic liposome includes the nucleic acid encoding the SRSF1 protein or a portion thereof.

Also described herein is a viral vector (e.g., lentiviral vector, retroviral vector, Adeno-Associated Virus vector, Adenovirus vector, Herpesvirus vector, etc.) including an expression construct including a nucleic acid encoding SRSF1 protein or a portion thereof. In some embodiments, the nucleic acid encodes a portion of the SRSF1 protein (e.g., RRM-1 and RRM-2).

Further described herein is a method for inhibiting viral replication in a subject. The method includes administering to the subject a composition including SRSF1 protein, a portion of a SRSF1 protein, or a nucleic acid encoding the SRSF1 protein or a portion thereof, in a therapeutically effective amount and a pharmaceutically acceptable carrier. In some embodiments, the portion of a SRSF1 protein includes RRM-1 and RRM-2. In some embodiments, the portion of a SRSF1 protein includes RRM-2. Typically, the virus is HIV-1 and the subject is a human. In the method, the SRSF1 protein or the portion of a SRSF1 protein can be conjugated to a CPP (e.g., Tat-CPP). The composition can further include at least one of: an endosomolytic agent (e.g., dfTat) and a CPP. In the method, a viral vector (e.g., lentiviral vector, retroviral vector, Adeno-Associated Virus vector, Adenovirus vector, Herpesvirus vector, etc.) or a cationic liposome can include the nucleic acid encoding the SRSF1 protein or a portion thereof. In a typical embodiment, administration of the composition inhibits viral transcription without affecting cell viability in the subject.

Additionally described herein is a method for the prevention, amelioration, or treatment of an HIV-1 infection in a subject. The method includes administering to the subject a composition including SRSF1 protein, a portion of a SRSF1 protein, or a nucleic acid encoding the SRSF1 protein or a portion thereof, in a therapeutically effective amount and a pharmaceutically acceptable carrier.

Yet further described herein is a kit for inhibiting viral replication in a subject. The kit includes a composition as described herein, instructions for use; and packaging. Unless otherwise defined, all technical terms used herein have the same meaning as commonly understood by one of ordinary skill in the art to which this invention belongs.

As used herein, “protein” and “polypeptide” are used synonymously to mean any peptide-linked chain of amino acids, regardless of length or post-translational modification, e.g., glycosylation or phosphorylation.

By the terms “SRSF1 protein” and “SRSF1 polypeptide” is meant an expression product of a SRSF1 gene such as the native human SRSF1 protein (UniprotKB Protein: Q07955 (SEQ ID NO:1); HGNC: 10780; Entrez Gene: 6426) or a protein that shares at least 65% (but preferably 75, 80, 85, 90, 95, 96, 97, 98, or 99%) amino acid sequence identity with the foregoing and displays a functional activity of a native SRSF1 protein. A “functional activity” of a protein is any activity associated with the physiological function of the protein. For example, functional activities of a native SRSF1 protein may include: regulation of RNA splicing, protein translation regulation, regulation of transcription from RNA polymerase II promoter, RNA binding, messenger RNA transport, messenger RNA processing, and alternative mRNA splicing.

“Purified,” as used herein, means separated from many other compounds or entities. A compound or entity (e.g., protein) may be partially purified, substantially purified, or pure. A compound or entity is considered pure when it is removed from substantially all other compounds or entities, i.e., is preferably at least about 90%, more preferably at least about 91%, 92%, 93%, 94%, 95%, 96%,

As used herein, the phrases “SRSF1 overexpression” and “overexpression of SRSF1” are used interchangeably to mean increased levels of SRSF1 mRNA and protein expression as compared to normal tissues.

By the term “gene” is meant a nucleic acid molecule that codes for a particular protein, or in certain cases, a functional or structural RNA molecule.

As used herein, a “nucleic acid” or a “nucleic acid molecule” means a chain of two or more nucleotides such as RNA (ribonucleic acid) and DNA (deoxyribonucleic acid).

By the terms “SRSF1 gene,” “SRSF1 polynucleotide,” and “SRSF1 nucleic acid” is meant a native human SRSF1-encoding nucleic acid sequence, e.g., the native human SRSF1 gene (RefSeq Accession: NM_006924 (SEQ ID NO: 2)) a nucleic acid having sequences from which a SRSF1 cDNA can be transcribed; and/or allelic variants and homologs of the foregoing. The terms encompass double-stranded DNA, single-stranded DNA, and RNA.

As used herein, the terms “cell penetrating peptide” and “CPP” mean any peptide sequence that facilitates the translocation of a cargo (e.g., peptide, polypeptide, nucleic acid) through a biological membrane. One example of a CPP is an HIV-1 Tat-derived peptide referred to herein as “Tat CPP” and “TatCPP” In a typical embodiment, the Tat CPP is composed of the YGRKKRRQRRR (SEQ ID NO:3) amino acid sequence derived from the Tat protein. Because the length and composition of the Tat CPP can vary, derivatives and mutants of SEQ ID NO:3 that vary in length and/or composition are encompassed by the invention herein.

As used herein, the term “vector” means a nucleic acid molecule capable of transporting another nucleic acid to which it has been linked. Vectors capable of directing the expression of genes to which they are operatively linked are referred to herein as “expression vectors.”

The terms “patient,” “subject” and “individual” are used interchangeably herein, and mean a mammalian (e.g., human) subject to be treated, diagnosed, and/or to obtain a biological sample from.

As used herein, “bind,” “binds,” or “interacts with” means that one molecule recognizes and adheres to a particular second molecule in a sample or organism, but does not substantially recognize or adhere to other structurally unrelated molecules in the sample. Generally, a first molecule that “specifically binds” a second molecule has a binding affinity greater than about 10⁸ to 10¹² moles/liter for that second molecule and involves precise “hand-in-a-glove” docking interactions that can be covalent and noncovalent (hydrogen bonding, hydrophobic, ionic, and van der waals).

The term “labeled,” with regard to a nucleic acid, peptide, polypeptide, cell, probe or antibody, is intended to encompass direct labeling of the nucleic acid, peptide, polypeptide, cell, probe or antibody by coupling (i.e., physically linking) a detectable substance to the nucleic acid, peptide, polypeptide, cell, probe or antibody.

When referring to a nucleic acid molecule or polypeptide, the term “native” refers to a naturally-occurring (e.g., a WT) nucleic acid or polypeptide.

As used herein, the terms “treatment” and “therapy” are defined as the application or administration of a therapeutic agent to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

As used herein, “sequence identity” means the percentage of identical subunits at corresponding positions in two sequences when the two sequences are aligned to maximize subunit matching, i.e., taking into account gaps and insertions. Sequence identity is present when a subunit position in both of the two sequences is occupied by the same nucleotide or amino acid, e.g., if a given position is occupied by an adenine in each of two DNA molecules, then the molecules are identical at that position. For example, if 7 positions in a sequence 10 nucleotides in length are identical to the corresponding positions in a second 10-nucleotide sequence, then the two sequences have 70% sequence identity. Sequence identity can be measured using any appropriate sequence analysis software.

When referring to mutations in a nucleic acid molecule, “silent” changes are those that substitute one or more base pairs in the nucleotide sequence, but do not change the amino acid sequence of the polypeptide encoded by the sequence. “Conservative” changes are those in which at least one codon in the protein-coding region of the nucleic acid has been changed such that at least one amino acid of the polypeptide encoded by the nucleic acid sequence is substituted with another amino acid having similar characteristics.

Although compositions, kits, and methods similar or equivalent to those described herein can be used in the practice or testing of the present invention, suitable compositions, kits, and methods are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1: Schematic representation of the pMtat(−) proviral clone.

FIG. 2A: Schematic representation of the SRSF1 deletion clones containing the RNA Recognition Motifs (RRMs) and the Arg-Ser rich domain (RS). FIG. 2B: Tat and SRSF1 expression in HEK293 cells transfected with SRSF1 and Tat coding plasmids. Proteins are detected with the anti-Tat, anti-SRSF1 and anti-Tubulin antibodies. The SRSF1 expressed from the pSRSF1 vector is tagged with a T7 epitope (T7-SRSF1) and migrates slower than the endogenous protein (endo-SRSF1). FIG. 2C: The T7-tagged SRSF1 proteins was detected utilizing an anti-T7 tag

FIG. 3A: Quantification of viral mRNA in a transient transfection system. HEK293. Cells were transfected with the proviral clone pMtat(−) and the indicated vector in the presence (+) or absence (−) of the Tat expression construct. Viral mRNA was quantified by qPCR (primers P1, P2), data were normalized for the α-Tubulin mRNA content of each sample and expressed as fold increase versus the pEGFP, Tat-transfection control. Tat expression level is shown for the cells transfected with pTat. FIG. 3B: Quantification of viral mRNA in the integrated provirus. HLM1 cells were transfected with the indicated expression vector in the presence (+) or absence (−) of Tat, viral mRNA was quantified by qPCR and expressed as fold increase versus the pEGFP, Tat-transfection control. Tat expression level is shown for the cells transfected with pTat. All data are represented as means±SEM.

FIG. 4A: Schematic representation of the pNL4-3 proviral clone. The relative position of the viral genes is indicated on the map on top. The main mRNAs, classified as unspliced (US), single spliced (SS) and multiply spliced (MS), are indicated. Multiple mRNAs coding for each viral gene are generated by the choice of several alternative 5′ and 3′ splice sites (10). Locations of the primers utilized in the qPCR assays to amplify the total viral mRNA (P1, P2) and specific for the gag/pol (P3, P4), env/vpu (P5, P6), rev (P9, P8), tat (P7, P8) and the multiple spliced mRNAs (tat, rev, nef) (P10, P11) are indicated. FIG. 4B: SRSF1 binding sites within the viral transcript. The sequences of four molecular clones were analyzed for consensus SRSF1 binding sites utilizing the ESEfinder 3.0. A high threshold stringency of 4 was chosen for the SRSF1 matrix analysis to reduce the number false positive and the significance of the output data. The position and score of each predicted SRSF1 binding site is plotted for each viral molecular clone in relation to the standard NL4-3 genome map. The location of the SRSF1 binding sites that have been experimentally characterized is indicated in relation to the NL4-3 genome (top).

FIG. 5A: Viral replication assay. Viral production was assayed as indicated in the schematics on the left. The amount of supernatant utilized for the assay was normalized for the amount of cells present in the plate. FIG. 5B: HEK293 cells were co-transfected with the viral expression clone pNL4-3 and the indicated SRSF1 plasmid. Inhibition of viral replication was defined as the: [Luciferase counts from TZM-bl infected with the supernatant of HEK293 transfected with pNL4-3 and the control pEGFP/Luciferase counts from TZM-bl infected with the supernatant of HEK293 transfected with pNL4-3 and the indicated SRSF1 clone].

FIG. 6A: Quantification of total viral mRNA from the transfected HEK293 cells by qPCR (primers P1, P2). The results are visualized as fold increase or decrease versus the control pEGFP transfection. FIG. 6B: Quantification of the single viral transcripts. Expression of the gag/pol, env, tat, rev and multiple spliced mRNAs relative to the total amount of viral transcript was calculated by normalizing the amount of specific transcripts for the total amount of viral mRNA in each sample and expressed as fold increase or decrease versus the control pEGFP transfection. All data are represented as means±SEM.

FIG. 7: Primary sequences alignments of the characterized SRSF1 binding sites in molecular clones from the B (NL4.3 and LAI.2), C (MJ4) and D (94UG114.1.6) viral subtypes. From top to bottom, the first sequence is SEQ ID NO:4, the second sequence is SEQ ID NO:5, the third sequence is SEQ ID NO:6, the fourth sequence is SEQ ID NO:7, the fifth and sixth sequences are SEQ ID NO:8, the seventh sequence is SEQ ID NO:9, the eighth sequence is SEQ ID NO:10, the ninth and tenth sequences are SEQ ID NO:11, the eleventh sequence is SEQ ID NO:12, and the twelfth sequence is SEQ ID NO:13.

FIG. 8: Quantification of viral replication and viral mRNAs for the NL4-3 molecular clone after expression of the SRSF1 constructs. Replication of the viral clone was assayed as described in FIG. 5(A). The relative amount of viral RNA was quantified as described in FIG. 6(A). The relative amounts of gag/pol and multiple spliced mRNAs were quantified as described in FIG. 6(B), normalized for the total viral mRNA content of each sample and expressed as fold increase or decrease versus the control pEGFP transfection. All data are represented as means±SEM.

FIG. 9: Quantification of viral replication and viral mRNAs for the LAI.2 molecular clone after expression of the SRSF1 constructs. Replication of the viral clone, the relative amount of viral RNA and the relative amounts of gag/pol and multiple spliced mRNAs were quantified as described in FIG. 8.

FIG. 10: Quantification of viral replication and viral mRNAs for the MJ4 molecular clone after expression of the SRSF1 constructs. Replication of the viral clone, the relative amount of viral RNA and the relative amounts of gag/pol and multiple spliced mRNAs were quantified as described in FIG. 8.

FIG. 11: Quantification of viral replication and viral mRNAs for the 94UG114.1.6 molecular clone after expression of the SRSF1 constructs. Replication of the viral clone, the relative amount of viral RNA and the relative amounts of gag/pol and multiple spliced mRNAs were quantified as described in FIG. 8.

FIG. 12A: Effects on cellular viability of SRSF1 deletion mutants. Cell viability was measured by quantifying cellular ATP production 72 hours post-transfection. Viability of the cells transfected with the SRSF1 clones is relative to that of the mock-transfected control. FIG. 12B: Effects on apoptosis of SRSF1 deletion mutants. Apoptotic events in transfected cells. Apoptosis in HEK293 cells transfected with the control and SRSF1 vectors was detected by visualizing the active apoptotic marker Caspase 9 72 hours post-transfection.

FIG. 13: Effects of the expression of SRSF1 deletion mutants on the the splicing of the CCDC115, C5ORF34, ENSA, FAS, HNRNPA1 cellular genes. SRSF1 target genes were analyzed by RT-PCR. Primers located in the exons flanking the alternatively spliced exon allow amplification of isoforms either including or excluding the regulated exon. For each gene we show the graphic output of the analysis (top) and the relative level of exon inclusion (bottom) in cells expressing the indicated SRSF1 or control plasmid 48 hours after transfection. The splicing of 3 genes is responsive solely to the expression of the full-length SRSF1 (*). 8 genes respond to both the wild type and RRM1+2 clones (**). 4 genes respond to the wild type, RRM1+2 and the RRM2 clones (***). All data are represented as means±SEM.

FIG. 14: Effects of the expression of SRSF1 deletion mutants on the the splicing of the HNRNPD, INO80E, LPIN1, MTMR14, NETO2 cellular genes. SRSF1 target genes were analyzed by RT-PCR as described in FIG. 13.

FIG. 15: Effects of the expression of SRSF1 deletion mutants on the the splicing of the SRSF1, SSFA2, TOP3B, PEA15, RPAIN cellular genes. SRSF1 target genes were analyzed by RT-PCR as described in FIG. 13.

FIG. 16A: Detection by western blot of SRSF1 and SRSF1 deletion mutants expression in HEK293 cells transfected with increasing amounts of SRSF1, deletion mutants and control plasmid DNA. Proteins were detected with antibodies anti-SRSF1, anti-T7 tag and anti-tubulin. All data are represented as means±SEM.16(B). FIG. 16B: Dose dependent response to SRSF1 expression. The proviral clone pNL4-3 was cotransfected with the indicated amount of each SRSF1 plasmid. Inhibition of viral replication was assayed as described in FIG. 5(A).

FIG. 17A: Cell viability in the dose dependent response to SRSF1 expression. HEK293 cell were cotransfected with the proviral clone pNL4-3 and the indicated amount of each SRSF1 plasmid. FIG. 17B: Apoptotic response in cells transfected with increasing amounts of SRSF1 and deletion mutants. Cell viability and apoptotic response were measured 72 hours past transfection. All data are represented as means±SEM.

FIG. 18: Localization of endogenous SRSF1 and EGFP tagged clones. Each panel (a) shows Nuclear DAPI staining, Each panel (b) shows endogenous SRSF1 was detected by immunofluorescence while tagged SRSF1-EGFP clones were detected by fluorescence microscopy. Each panel (c) shows merged (a) and (b) showing localization of the endogenous and tagged proteins within the nuclei and cytosol.

FIG. 19A: The schematic summarizes the viral replication assay. H9 cells were infected with NL4-3 virus at a high MOI (≧100). The infected cells were than transfected with the SRSF1-EGFP clones, sorted for EGFP expression and viral replication assayed in the following days as summarized in the schematics on the left. FIG. 19B: Inhibition of viral replication in H9 cells. Inhibition of viral replicaiton was measured as described as described in FIG. 5(A) for four consecutive days following cell sorting. FIG. 19C: Cell viability of the infected cells transfected with the EGFP-tagged SRSF1 clones was measured by quantifying cellular ATP production five days post-transfection. Viability of the cells transfected with the SRSF1-EGFP clones is relative to that of the EGFP-transfected control. All data are represented as means±SEM.

FIG. 20A: Schematic representation of the pLTR-Xm-LR reporter gene carrying a luciferase gene under the control of an IRES translation sequence and the LTR promoter. FIG. 20B: SRSF1 inhibits viral transactivation of the pLTR-Xm-LR reporter. HEK293 cells were co-transfected with pLTR-Xm-LR, SRSF1 or its deletion clones and a Tat expression plasmid as indicated. FIG. 20C: SRSF1 inhibits viral transactivation in the full length viral clone pMtat(−), which does not express Tat in HEK293 cells co-transfected as indicated in FIG. 20(B).

FIG. 21A: SRSF1 and Tat compete for the binding of overlapping sequences within TAR. RNA affinity chromatography (RAC) assays were setup with bait RNAs containing the wild-type (TARWT) and mutated (TARM) TAR sequences. The RNA substrates were incubated with 100 ng of recombinant Tat or purified SRSF1 in separate reactions. FIG. 21B: Tat and SRSF1 compete for binding onto TAR. RAC assays were set up with the wild-type TAR sequence as bait, and either 100 ng of recombinant Tat and increasing amounts of purified SRSF1 or increasing amounts of Tat and 100 ng of SRSF1.

FIG. 22: Model of the control of HIV-1 transcription by the interplay between SRSF1 and Tat.

FIG. 23: Schematic representation of the TatCPP:GB1:RRMs chimeras and flow chart of the experimental procedures that will be utilized to purify, validate and analyze the antiviral activity of the chimeric proteins.

FIG. 24: Inhibition of viral replication obtained by transfecting expressing HIV-1 with vectors expressing the proteins described in FIG. 2(A), FIG. 23 and FIG. 25.

FIG. 25: Summary of the clones to be utilized for the delivery of chimeric proteins with increased specificity for HIV-1 infected lymphocytes.

FIG. 26: Schematic representation of the GFP-tagged SRSF1 clones and summary of their intracellular localization as observed in HEK293 cells.

FIG. 27: HEK293 cells were transfected with the clones represented in FIG. 26. Each panel labeled (a) is Nuclear DAPI staining, each panel labeled (b) is endogenous SRSF1 was detected by immunofluorescence while tagged SRSF1-EGFP clones were detected by fluorescence microscopy. Each panel labeled (c) is merged (a) and (b) showing localization of the endogenous and tagged proteins within the nuclei and cytosol.

FIG. 28A: Schematic representation of the proposed deletion library to locate the NLS present in the RRM1+2 clone. FIG. 28B: Schematic flow chart to validate the activity of the NLS-RRM2 chimeras.

FIG. 29: The CPP-tagged SRSF1 proteins are delivered to a HEK293 cell line by short (1 hr) co-incubation in the culture media and visualized with antibodies against the His Tag or DAPI staining. Each panel labeled (b) is visualization with antibodies against the His Tag. Each panel labeled (a) shows DAPI staining. Each panel labeled (c) is merged. The localization pattern is comparable to the one of the wild type SRSF1 and the deletion mutant containing the two RRMs delivered by transfection of an expression vector and visualized with an antibody against a T7-Tag.

FIG. 30A: The CPP-tagged SRSF1 proteins are delivered to a HEK293 cell line by short (1 hr) co-incubation in the culture media. Western blot analysis show that the recombinant proteins reached an intracellular concentration upward to 20 fold the one of the endogenous SRSF1. FIG. 30B: Delivery of the CPP-tagged SRSF1 in HEK293 show minimal cellular toxicity.

### DETAILED DESCRIPTION

Described herein are compositions including a therapeutically effective amount of SRSF1 protein, a portion of a SRSF1 protein, or a nucleic acid encoding the SRSF1 protein or a portion thereof, for inhibiting replication of a virus in a subject (e.g., a human patient) infected with the virus or at risk of being infected with the virus and a pharmaceutically acceptable carrier. Methods of using these compositions, and kits including these compositions, are also described herein. The experimental results described herein demonstrate the therapeutic utility of SRSF1, a cellular RBP, that was shown to inhibit the expression of the HIV genome by binding a series of sequences within the viral transcript. The data indicate that the expression of a SRSF1 fragment (a portion of SRF1), composed of the protein's two RBDs, can inhibit viral replication up to 3,000 fold in several viral subtypes with no effect on cell viability. Described herein is the development of novel therapies for AIDS based on the delivery of SRSF1, single protein domains (portions) derived from SRSF1, and compounds mimicking the activity of such domains and the development of reagents for the mechanistic study of SRSF1 and other proteins' activity in viral replication and possibly latency.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; The Condensed Protocols From Molecular Cloning: A Laboratory Manual, by Joseph Sambrook et al., Cold Spring Harbor Laboratory Press, Cold Spring Harbor, N.Y., 2006; and Current Protocols in Molecular Biology, ed. Ausubel et al., Greene Publishing and Wiley-Interscience, New York, 1995 (with periodic updates). Conventional methods of gene transfer and gene therapy may also be adapted for use in the present invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; Viral Vectors for Gene Therapy: Methods and Protocols, ed. Otto-Wilhelm Merten and Mohammed Al-Rubeai, Humana Press, 2011; and Nonviral Vectors for Gene Therapy: Methods and Protocols, ed. Mark A. Findeis, Humana Press, 2010. Methods for constructing and using viral vectors are known in the art (see, e.g., Miller and Rosman, BioTechniques 1992, 7:980-990). Cationic liposomes and methods of producing and using for gene delivery are known and are described in Simões et al., Expert Opin Drug Deliv 2005 (2(2):237-254).

### Vectors, Compositions and Methods for Inhibiting Viral Replication

Compositions described herein for inhibiting viral replication include a therapeutically effective amount of SRSF1 protein, a portion (e.g., a domain) of a SRSF1 protein, or a nucleic acid encoding the SRSF1 protein or a portion thereof, for inhibiting replication of a virus in a subject (e.g., a human) infected with the virus or at risk of being infected with the virus and a pharmaceutically acceptable carrier. Inhibiting viral replication includes inhibiting viral transcription and inhibiting processing of viral messenger RNAs. In the experiments described herein, HIV-1 replication was inhibited using SRSF1 and portions (e.g., domains) thereof.

A composition for inhibiting replication of a virus in a subject can include SRSF1 protein or a portion thereof. A composition can alternatively include a nucleic acid that encodes SRSF1 protein or a portion thereof. SRSF1 is composed of two RNA Recognition Motif (RRM) RNA-binding domains (RBDs), which interact with specific RNA sequences, and an RS (arginine/serine-rich) carboxy-terminal domain, which is required for protein-protein interaction. In some embodiments, the entire (full-length) SRSF1 protein (e.g., wild-type or mutated) can be used. In other embodiments, a portion or portions of the SRSF1 protein can be used. A portion can include one or more fragments or domains of SRSF1. For example, the region of SRSF1 containing domains RRM-1 and RRM-2 can include amino acids 16 to 195 of SRSF1 (16 to 195 of SEQ ID NO:1, Accession No.: UniprotKB Protein Q07955) can be used. Truncated and/or mutated versions of the SRSF1 protein region containing RRM-1 and RRM-2 can be used in the invention. As another example, RRM-2 is used. RRM-1 includes amino acids 16 to 91 of SRSF1 (16 to 91 of SEQ ID NO:1, Accession No.: UniprotKB Protein Q07955). Truncated and/or mutated versions of RRM-1 can be used in the invention. RRM-2 includes amino acids 121-195 of SRSF1 (121-195 of SEQ ID NO:1, Accession No.: UniprotKB Protein Q07955). Truncated and/or mutated versions of RRM-2 can be used in the invention. A series of SRSF1 deletion mutants were created, expressed and tested as described in the Examples below. See SEQ ID NO:s 17-19 in Example 4 for examples of RRM1+2, RRM1 and RRM2 sequences.

In some embodiments, the SRSF1 protein or portion thereof is conjugated to a CPP, e.g., to Tat. In these embodiments (e.g., Tat-CPP), the CPP is fused to the SRSF1 protein or portion thereof. Alternatively, the SRSF1 protein or portion thereof can be administered to cells in combination with a CPP. For example, a composition can include a therapeutically effective amount of SRSF1 protein or a portion of a SRSF1 protein, as well as the CPP. In such an embodiment, the CPP may be incubated with the SRSF1 protein or portion thereof before delivering to cells. The SRSF1 protein or portion thereof can be fused with, tagged with, and/or conjugated to additional peptide sequences such as a His tag for purification, a GB1 domain for solubility, etc. Any suitable such peptide sequences having a desirable function or characteristic can be used. A CPP can also be utilized to deliver nucleic acids. In such embodiments, the CPP can be either covalently linked to a nucleic acid(s) or coincubated with a nucleic acid(s).

In other embodiments, the SRSF1 protein or portion thereof is delivered to cells in the presence of an endosmolytic agent. Any suitable endosmolytic agent can be used, e.g., dfTat (Erazo-Oliveras et al., 2014. Nat Methods 11:861-867). In some embodiments, both a CPP and an endosmolytic agent may be included in a composition for optimizing delivery of the SRSF1 protein (or portion thereof) into cells by endosomal uptake and subsequent endosomal release of the SRSF1 protein (or portion thereof) within the transduced cell. dfTat is an agent that can be co-incubated with the cargo (the SRSF1 protein or portion thereof) before delivery to the cells. dfTat can work without the cargo fused to a CPP, but typically works even better when the cargo is fused to a CPP. It works in a similar way as a liposome to enhance or facilitate delivery. Examples of dfTat-containing compositions include a composition including dfTat+SRSF1, and a composition including dfTat+TatCPP:SRSF1 (where TatCPP:SRSF1 is a fusion protein).

Typically, the compositions are delivered to appropriate target cells in the subject (e.g., human patient). A target cell is any cell that is infected or may become infected by the HIV-1 virus. Target cells include but are not limited to any cell that expresses on its surfaces the receptors recognized by the HIV-1 virus. Such receptors are the product of the CD4 gene (HGNC 1678) in combination with either the product of the gene CCR5 (HGNC 1606) or CXCR4 (HGNC 2561). Examples of cells that expresses the CD4 gene in combination with either the CCR5 or the CXCR4 gene include but are not limited to: CD4++T cells, macrophages, dendritic cells, microglial cells. Cells that do not express the CD4 and CCR5 or CD4 and CXCR4 gene products and can be infected by HIV-1 through not well characterized mechanisms include, but are not limited to, fibroblasts, astrocites, hepatocites.

Many vectors useful for introducing exogenous nucleic acids into target cells (e.g., mammalian cells) are available. The vectors may be episomal, e.g., plasmids, virus-derived vectors such as those from cytomegalovirus, adenovirus, herpesvirus, retrovirus, Alphavirus, AAV, lentivirus etc., or may be integrated into the target cell genome, through homologous recombination or random integration, e.g., retrovirus-derived vectors such MMLV, HIV-1, ALV, etc. In one embodiment, the SRSF1 protein or portion of a SRSF1 protein is encoded by a nucleic acid contained within a viral vector. In such an embodiment, recombinant viral vectors or recombinant virions (particles) containing the viral vector are administered to the subject. Viruses are naturally evolved vehicles which efficiently deliver their genes into host cells and therefore are desirable vector systems for the delivery of therapeutic nucleic acids. Preferred viral vectors exhibit low toxicity to the host cell and produce/deliver therapeutic quantities of the nucleic acid of interest (in some embodiments, in a tissue-specific manner). Such recombinant virions may be pseudotyped, and/or engineered with cell- or tissue-specific tropism. In some embodiments, the viral vectors of the invention are replication defective, that is, they are unable to replicate autonomously in the target cell. In one embodiment of the invention, lentiviral vectors can be used for the direct delivery and sustained expression of a transgene (e.g., a nucleic acid encoding SRSF1 protein or portion thereof) in several tissue types. This subtype of retroviral vector can efficiently transduce dividing and nondividing cells in these tissues, and maintain long-term expression of the gene of interest (for a review, see, Naldini, Curr. Opin. Biotechnol. 1998, 9:457-63; Zufferey, et al., J. Virol. 1998, 72:9873-80). Lentiviral packaging cell lines are available and known generally in the art (see, e.g., Kafri, et al., J. Virol., 1999, 73: 576-584).

Many nonviral techniques for nucleic acid delivery can be used for delivering a nucleic acid encoding SRSF1 protein or portion of a SRSF1 protein to cells. One example is the use of carbon nanotubes for DNA/RNA delivery. The use of carbon nanotubes as gene delivery vectors is known in the art. Methods of production and use thereof are described, for example, in Ramos-Perez et al., Methods Mol Biol. 2013; 1025:261-268. Electroporation is another example, and is widely known in the art. Cationic lipid DNA delivery is another example. Compositions of synthetic cationic lipids, which can be used to prepare liposomes for in vivo transfection of a vector carrying a transgene (e.g., an SRSF1 gene or portion thereof), are described in Feigner et. al., Proc. Natl. Acad. Sci. USA 1987, 84:7413-7417; Feigner and Ringold, Science 1989, 337:387-388; Mackey, et al., Proc. Natl. Acad. Sci. USA 1988, 85:8027-8031; and Ulmer et al, Science 1993, 259:1745-1748. Useful lipid compounds and compositions for transfer of nucleic acids are described, e.g., in PCT Publications No. WO 95/18863 and WO96/17823, and in U.S. Pat. No. 5,459,127. In some embodiments, a nucleic acid encoding SRSF1 protein or portion of a SRSF1 protein is RNA, e.g., mRNA. Methods of delivering mRNA to cells involving the synthesis of modified mRNA incorporating nucleotide analogs, including but not limited to 2-thiouridine and 5-methylcytidine, to improve the mRNA stability and avoid cellular inflammatory reactions, are known and are described, for example, in Warren et al. 2010, Cell Stem Cell 7: 618-630 and Kormann et al. 2011, Nat Biotechnol 29: 154-157.

With regard to delivery of a therapeutically effective amount of SRSF1 protein, a portion of a SRSF1 protein, or a nucleic acid encoding the SRSF1 protein or a portion thereof, any suitable delivery methods and vehicles may be used. Nucleic acids, proteins and viral vectors can be delivered to cells using extracorporeal delivery to T cells (or other cell types). An extracorporeal cell-based therapeutic device and delivery system is described, for example, in US patent application no. US2011/190679 and Bieber et al. (2013); Sci Rep 3:1538.

Methods of inhibiting replication of a virus in a subject include administering to the subject a composition including a therapeutically effective amount of SRSF1 protein, a portion of a SRSF1 protein, or a nucleic acid encoding the SRSF1 protein or a portion thereof, for inhibiting replication of a virus in the subject (e.g, a subject infected with the virus or at risk of infection) and a pharmaceutically acceptable carrier. The methods include administration of any of the compositions described herein. In a typical embodiment, administration of a composition inhibits viral transcription without affecting cell viability in the subject (i.e., viability of the subject's own cells). In some embodiments, administration of the composition to the subject results in prevention, amelioration, or treatment of an HIV-1 infection (e.g., alleviation or mitigation of AIDS symptoms or pathology) in a subject.

Any suitable methods of administering such a composition to a subject may be used. In these methods, the compositions can be administered to a subject by any suitable route, e.g., systemically by intravenous injection, directly to a target site, parenterally, orally, etc. The compositions may be administered directly to a target site by, for example, surgical delivery to an internal or external target site, or by catheter to a site accessible by a blood vessel. If administered via intravenous injection, the compositions may be administered in a single bolus, multiple injections, or by continuous infusion (e.g., intravenously, by peritoneal dialysis, pump infusion). For parenteral administration, the compositions are preferably formulated in a sterilized pyrogen-free form. As indicated above, the compositions described herein may be in a form suitable for sterile injection. To prepare such a composition, the suitable active therapeutic(s) (e.g., a therapeutically effective amount of SRSF1 protein, a portion of a SRSF1 protein, a nucleic acid encoding the SRSF1 protein or a portion thereof, a vector encoding same) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the therapeutics is only sparingly or slightly soluble in water, a dissolution enhancing or solubilizing agent can be added, or the solvent may include 10-60% w/w of propylene glycol or the like. The compositions described herein may be administered to mammals (e.g., rodents, humans, nonhuman primates, canines, felines, ovines, bovines) in any suitable formulation according to conventional pharmaceutical practice (see, e.g., Remington: The Science and Practice of Pharmacy (20th ed.), ed. A. R. Gennaro, Lippincott Williams & Wilkins, (2000) and Encyclopedia of Pharmaceutical Technology, eds. J. Swarbrick and J. C. Boylan, Marcel Dekker, New York (1988-1999), a standard text in this field, and in USP/NF). A description of exemplary pharmaceutically acceptable carriers and diluents, as well as pharmaceutical formulations, can be found in Remington: supra. Other substances may be added to the compositions to stabilize and/or preserve the compositions.

The therapeutic methods described herein in general include administration of a therapeutically effective amount of the compositions described herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof (e.g., HIV-1 infection, AIDS). Determination of those subjects “at risk” can be made by any objective or subjective determination by a diagnostic test or opinion of a subject or health care provider.

### Effective Doses

The compositions described herein are preferably administered to a mammal (e.g., human) in an effective amount, that is, an amount capable of producing a desirable result in a treated mammal (e.g., inhibiting HIV-1 replication). Such a therapeutically effective amount can be determined according to standard methods. Toxicity and therapeutic efficacy of the compositions utilized in methods of the invention can be determined by standard pharmaceutical procedures. As is well known in the medical and veterinary arts, dosage for any one subject depends on many factors, including the subject's size, body surface area, age, the particular composition to be administered, time and route of administration, general health, and other drugs being administered concurrently. A delivery dose of a composition as described herein is determined based on preclinical efficacy and safety.

### Kits

Described herein are kits for inhibiting viral replication in a subject. A typical kit includes a composition including a pharmaceutically acceptable carrier (e.g., a physiological buffer) and a therapeutically effective amount of SRSF1 protein, a portion of a SRSF1 protein, or a nucleic acid encoding the SRSF1 protein or a portion thereof; and instructions for use. Such kits also typically include a container and packaging. Instructional materials for preparation and use of the compositions and vectors described herein are generally included. While the instructional materials typically include written or printed materials, they are not limited to such. Any medium capable of storing such instructions and communicating them to an end user is encompassed by the kits herein. Such media include, but are not limited to electronic storage media (e.g., magnetic discs, tapes, cartridges, chips), optical media (e.g., CD ROM), and the like. Such media may include addresses to internet sites that provide such instructional materials.

### Data and Analysis

Use of the compositions, methods, and kits described herein may employ conventional biology methods, software and systems. Useful computer software products typically include computer readable medium having computer-executable instructions for performing logic steps of a method. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001). See U.S. Pat. No. 6,420,108.

The compositions, methods, and kits described herein may also make use of various computer program products and software for a variety of purposes, such as reagent design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170. Additionally, the embodiments described herein include methods for providing data (e.g., experimental results, analyses) and other types of information over networks such as the Internet.

### EXAMPLES

The present invention is further illustrated by the following specific examples. The examples are provided for illustration only and should not be construed as limiting the scope of the invention in any way.

### Example 1

The SRSF1 RNA Recognition Motifs are strong inhibitors of HIV-1 replication (Sean Paz, Michael L. Lu, Hiroshi Takata, Lydie Trautmann and Massimo Caputi (2015) The SRSF1 RNA Recognition Motifs of are strong inhibitors of HIV-1 replication. J. Virology. 89(12):6275-86)

Replication of the integrated HIV-1 genome is tightly regulated by a combination of host and viral factors. Interactions between viral sequences, cellular and viral proteins are required to express the viral genome and alteration of the mechanisms regulating transcription, splicing and export of the viral transcripts can dramatically affect HIV-1 infectivity and pathogenesis.

The integrated provirus is transcribed into a single pre-mRNA from a promoter located within the 5′ long terminal repeat (LTR) of the viral genome through RNA polymerase II (RNAPII) and a combination of basal and promoter specific factors. The viral protein, Tat, stimulates transcription elongation by binding to a structured RNA element (transactivation responsive region, TAR), located at the 5′ ends of all nascent HIV-1 transcript and triggering the recruitment of the P-TEFb complex. The P-TEFb complex is composed of cellular cyclin T1 (CycT1) and the cyclin-dependent kinase 9 (CDK9). P-TEFb activates viral transcription by promoting the release of the NELF and DRB sensitivity inducing factor (DSIF) transcriptional pausing complex and phosphorylation of the C-terminal domain (CTD) of RNAP II to facilitate elongation of the viral transcript.

The single viral transcript undergoes a complex series of splicing events to generate over 40 mRNA isoforms, thus, the same viral protein is encoded by multiple mRNAs that vary for their 5′ and 3′ untranslated regions. Spliced viral mRNAs can be classified in a group of approximately 4 kb in length, coding for the Env, Vpu, Vpr and Vif proteins, and a group of approximately 2 kb in length, coding for the Tat, Rev and Nef proteins. Furthermore, approximately 50% of the viral pre-mRNAs leave the nucleus without being spliced. The unspliced 9 kb mRNA encodes for the Gag and Gag-Pol polyprotein and is packaged within the nascent virions as viral genome. The complex splicing regulation of the viral transcripts is carried out by several cellular factors, which interact with partially characterized cis-acting elements distributed throughout the genome and selectively enhance or inhibit the use of specific splice sites.

It was recently shown that SRSF1, an RNA binding protein (RBP) member of the serine/arginine (SR) proteins family, can inhibit Tat transactivation by directly competing for its binding onto TAR (Sean Paz, Adrian R. Krainer and Massimo Caputi. (2014) Nucleic Acids Res. 42:13812-13823). SR proteins are key regulators of gene expression, highly conserved and widely expressed in eukaryotes. SRSF1 and other members of this protein family regulate the assembly of the splicing machinery, integrate multiple steps in RNA metabolism and have been shown to modulate RNAPII activity. SRSF1 binds to splicing regulatory sequences within the viral transcript and modulates the splicing of subgenomic clones in-vitro and ex-vivo. Nevertheless, given the presence of multiple putative binding sites for SRSF1 throughout the viral genome and the high mutation rate in the primary sequences among different viral isolates the overall contribution of this protein to viral replication, in the context of the full-length virus, is unclear.

The role of SRSF1 in viral gene expression and replication was analyzed utilizing molecular clones from different viral subtypes. The results described herein showed that overexpression of SRSF1 strongly inhibits viral transcription, splicing and replication of viruses from the B, C and D subtypes. Furthermore, it was found that the SRSF1 RNA binding domains (RBDs) alone can down-regulate viral replication by over 2,000 fold without altering cell viability and apoptosis. Taken together, these results demonstrate the therapeutic utility of SRSF1 and its RBDs.

### Materials and Methods

Plasmids and Cells

The SRSF1 deletion mutants have been previously described (Caceres et al., 1997, The Journal of Cell Biology 138:225-238). The EGFP tagged SRSF1 clones were obtained by cloning the SRSF1 coding sequences upstream the EGFP gene in the pEGFP-C1 vector (Clontech). HIV-1 molecular clones, pNL4.3 contributed by Dr. Malcolm Martin (Adachi et al., 1986, Journal of Virology 59:284-291), pMtat(−) contributed by Dr. Reza Sadaie (Sadaie et al., 1988, Science 239:910-913) pLAI.2 contributed by K. Peden (Peden et al., 1991, Virology 185:661-672), pMJ4 contributed by Dr. Ndung'u (Ndung'u et al., 2001, Journal of Virology 75:4964-4972) and p94UG114.1.6 contributed by Dr. Hahn (Gao et al., 1998, Journal of Virology 72:10234-10241) were obtained through the AIDS Research and Reference Reagent Program, Division of AIDS, NIAID, NIH.

HEK293 cells were maintained at below 80% confluence. Cells were transfected in 24 well plates with Lipofectamine 2000 (Life Technologies) 0.2 μg of virus coding plasmid and 0.2 μg of the SRSF1 or control expression plasmids. Proteins expressed were analyzed utilizing the antibodies anti-SRSF1 (provided by Dr. A. R. Krainer, Cold Spring Harbor Laboratories), Tat (obtained through the NIH AIDS Reagent Program, Division of AIDS, NIAID, NIH: Antiserum to HIV-1 Tat from Dr. Bryan Cullen), GFP (B-2, Santa Cruz Biotech), β-Tubulin (Sigma) and T7-Tag (Sigma). HLM1 (obtained through the NIH AIDS Reagent Program, Division of AIDS, NIAID, NIH: HLM30 Cells from Dr. Reza Sadaie) were transfected by electroporation utilizing a GenePulserll (BioRad).

In-Vitro Infection Assay

H9 cells (obtained through the NIH AIDS Reagent Program, Division of AIDS, NIAID, NIH: H9 from Dr. Robert Gallo) were infected at high MOI (≦100) with virus generated by collecting the supernatant from HEK293 cells transfected with the proviral clone pNL4-3. Four days past-infection cells were transfected with the EGFP tagged clones by electroporation utilizing a GenePulserll (BioRad). 24 hours post-transfection, EGFP expressing cells were sorted using a BD Facs Aria II cell sorter. Cells displayed peak EGFP signal were gated and collected. Post-sorting, the cells were collected and plated in 96 well-plates at a concentration of 50,000 cells/well in triplicates. Virus was collected every 24 hours for the following four days and quantified utilizing TZM-bl cells.

RNA Extraction and Transcripts Analysis.

Total RNA was extracted 48 after transfection with the Total RNA Isolation Kit (Agilent) and DNase treated with Turbo DNase (Ambion). RNA was reverse transcribed utilizing a random pd(N)6 primer and Superscript II RT (LifeTechnologies). Quantitative PCR analysis of the viral transcripts was obtained as previously described. Each sample was normalized for the relative content in the housekeeping genes GAPDH and Tubulin. qPCR was performed utilizing a Stratagene Mx3005P real time PCR system, SYBR green dye and analyzed with MxPro V3.0 software. Each assay was carried out with a minimum of three independent transfections while qPCR assays were carried out in duplicates. Data are represented as means±SEM. Alternative splicing events in cellular genes were quantified by semi-quantitative RT-PCR assays utilizing the primer sets as previously described. To avoid amplification bias 28 cycles were utilized for each PCR assay. PCR products were analyzed and quantified utilizing a 2100 Bioanalyzer (Agilent Technologies) and 2100 Expert Software. Data are represented as means±SEM.

Cellular Assays

The viral replication assay was carried out utilizing TZM-bl cells seeded 24 hours before infection in 96 well plates at 50% confluence in 200 μL of D-MEM supplemented with 8% fetal calf serum and gentamicin. Supernatant collected from the HEK293 cells 72 hours after the transfection carried out with the proviral constructs was utilized to infect the TZM-bl cells. At 48 hours post infection cells were lysed and luciferase expression was assayed and quantified utilizing a BMG PolarStar Omega reader. Each assay was carried out with a minimum of three independent transfections. TZM-bl infections from each independent assay were carried out in triplicates. Luciferase data were analyzed utilizing the MARS data analysis software. Cell viability was measured in HEK293 cells 72 hours post transfection utilizing the CellTiterGlo (Promega) ATP Production Assay. The assay was performed in HEK293 cells 72 hours post transfection and in H9 cells 5 days post transfection. Apoptotic events were detected utilizing the FAM FLICA Caspase 9 Apoptosis Detection Kit (Marker Gene Technologies). Data are represented as means±SEM. The assay was performed in HEK293 cells 72 hours post transfection.

### Results

SRSF1 Inhibits Tat Transactivation

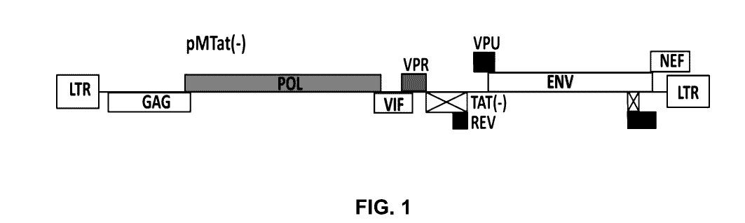

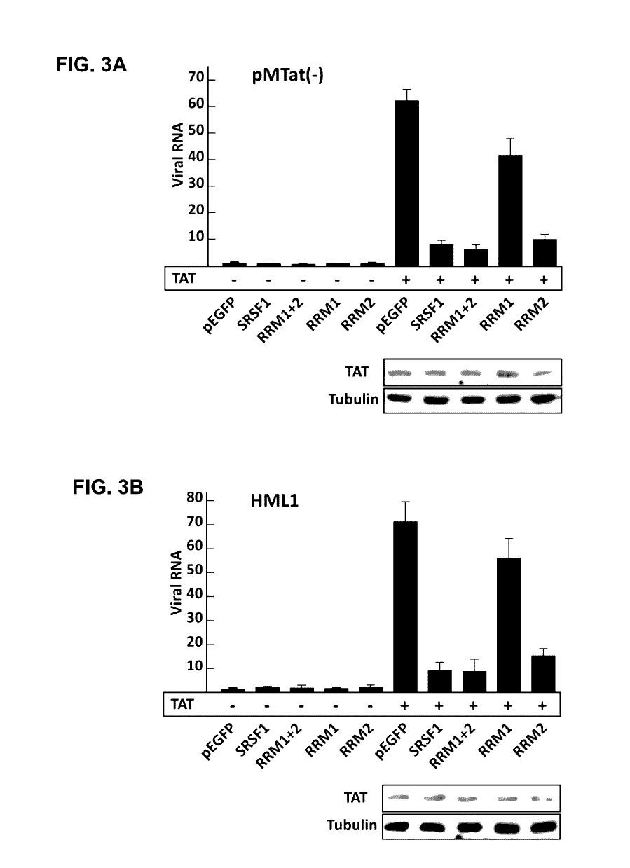

The cellular machineries regulating the transcription and processing of eukaryotic RNAs are intimately coupled. Components of the RNAPII transcription complex, modulate the processing of the nascent pre-mRNA and, in some cases, processing feeds back to regulate transcription, therefore it is important to study the regulation of viral gene expression within the contest of the full length virus. The role of SRSF1 in HIV transcription was studied in the context of the full-length virus. The proviral clone pMtat(−), which contains a full-length viral genome but lacks a functional copy of the Tat gene (FIG. 1) (Sadaie et al., 1988, Science 239:910-913), was used. HEK293 cells were co-transfected with the proviral vector pMtat(−) and expression clones for Tat, SRSF1, SRSF1 deletion mutants or the control EGFP. Tat increased the amount of viral transcripts by over 60-fold, whereas SRSF1 overexpression reduced transactivation to 8-fold (FIG. 3(A)).

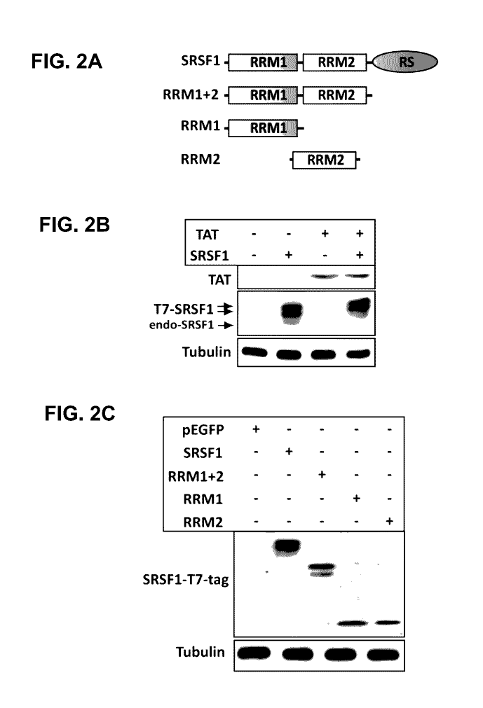

SRSF1 is composed of two RNA Recognition Motif (RRM) RNA-binding domains (RBDs), which interact with specific RNA sequences, and an RS (arginine/serine-rich) carboxy-terminal domain, which is required for protein-protein interaction but does not appear to affect the RNA binding specificity of the protein. Previous results indicated that the ability of SRSF1 to inhibit transactivation was solely dependent on its binding to TAR. To determine if this was also true in the context of the full-length virus, a series of SRSF1 deletion mutants (FIGS. 2(A), 2(C)) were expressed in the absence or presence of Tat.

The mutant carrying a deletion of the protein-interacting RS domain reduced transactivation to only 6 fold, which is lower than the one observed utilizing the wild type protein (8 fold). Expression of a mutant clone carrying only RRM2 decreased transactivation with efficiency similar to the wild type protein (10 fold) while RRM1 reduced transactivation by less than 50%. These data are consistent with previous observations suggesting an inhibition mechanism solely dependent on the RNA binding specificity of SRSF1 for a sequence overlapping the Tat binding site onto TAR and with findings suggesting that the RNA binding activity of SRSF1 is mostly dependent on RRM2 (Clery et al., 2013, Proceedings of the National Academy of Sciences USA 110:E2802-2811).

One of the limitations of utilizing a transient transfection system to study the mechanism of viral replication is the presence of multiple non-integrated copies of the viral genome. Although, the transcription machinery appears to similarly regulate the integrated and non-integrated viral genome, it is conceivable that the transcriptional effect of SRSF1 might differ in the course of the natural infection, with cells carrying a single copy of the genome integrated in the chromosomal DNA. Results obtained using HLM1 cells, which carry a single copy of the integrated pMtat(−) (FIG. 3(B)), validate the data obtained with the transfection of pMtat(−) and indicate that the transient transfection system utilized can be adopted to test the effects of SRSF1 on viral production.

SRSF1 RBDs Inhibit Viral Production.

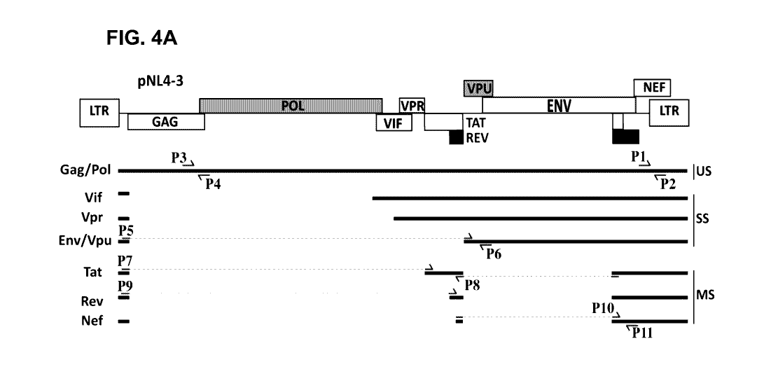

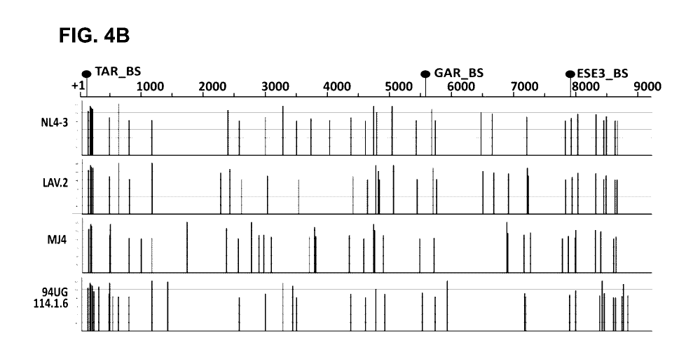

SRSF1 exerts a key role in the regulation of viral splicing. Previous work characterized splicing regulatory sequences bound by SRSF1 that modulate selection of specific splice sites by the cellular splicing machinery. Furthermore, in-silico analysis of the viral transcript performed using the ESEfinder 3.0 (Cartegni et al., 2003, Nucleic Acids Research 31:3568-3571), a SR protein functional binding site prediction matrix, indicates that several putative high-affinity SRSF1 binding sites are present throughout the viral genome (FIG. 4(B)), revealing a pervasive role for this splicing factor in the regulation of the viral pre-mRNA.

Two mechanisms have been proposed to explain SRSF1 role in splicing regulation (Long J C, Caceres J F, 2009, The Biochemical Journal 417:15-27). In one model, SRSF1 recruits, via protein-protein interactions with the RS domain, essential component of the basal splicing machinery onto 5′ and 3′ splice sites. In the second model, SRSF1 activates splicing by competing with and inhibiting the binding of splicing repressors in the proximity of the regulated splice site. Furthermore, this protein has also been shown to inhibit the splicing of a number of cellular exons although the mechanism of action is unclear.

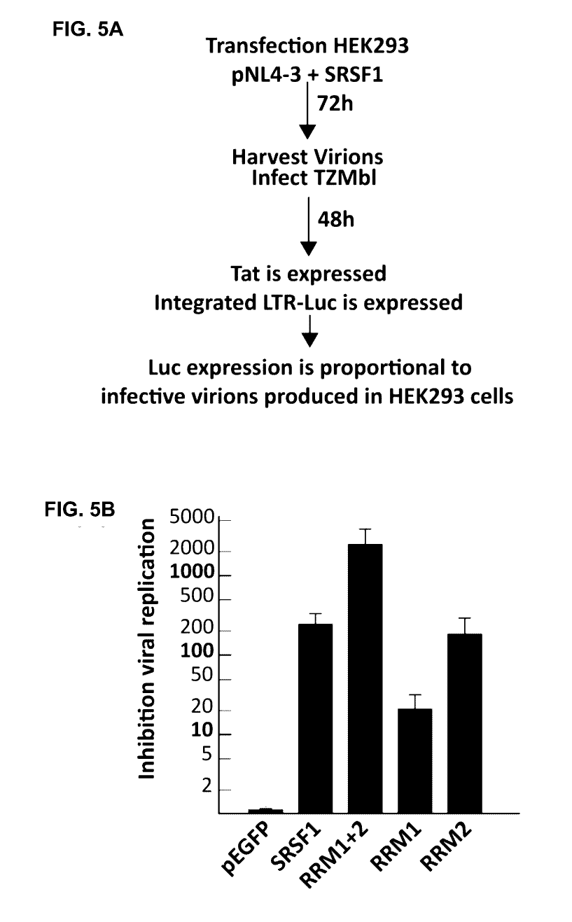

Experiments were undertaken to determine how the wild type and its single domain deletion mutants might affect the biogenesis of the viral mRNAs and viral replication at large. To this end, a viral production assay was utilized. HEK293 cells were co-transfected with the proviral vector pNL4-3, which codes for a replication competent copy of the viral genome, and the SRSF1 full length or RBD domain constructs. Transfection of HEK293 cells is highly efficient, thus ensuring co-expression of the proviral and SRSF1 constructs. Viral production was analyzed by collecting the supernatant from the transfected HEK293 cells and infecting TZM-bl cells, which contain a copy of the luciferase gene under control of the HIV-1 LTR promoter. Infected cells express the viral protein Tat, which in turn activates the viral LTR promoter. Thus, the number of infective viral copies are reliably quantified by the activity of the luciferase gene product. Overexpression of SRSF1 decreased viral production by over 200 fold. Surprisingly, a mutant lacking the RS domain exhibited decreased production by over 2,000 fold while the result obtained following expression of the RRM2 alone was comparable to the wild type (FIG. 5(C), 5(B)).

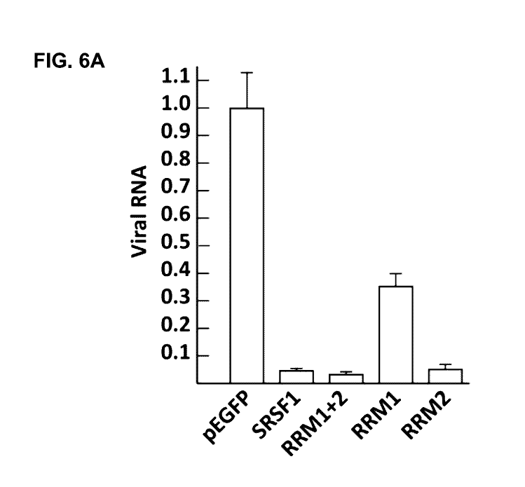

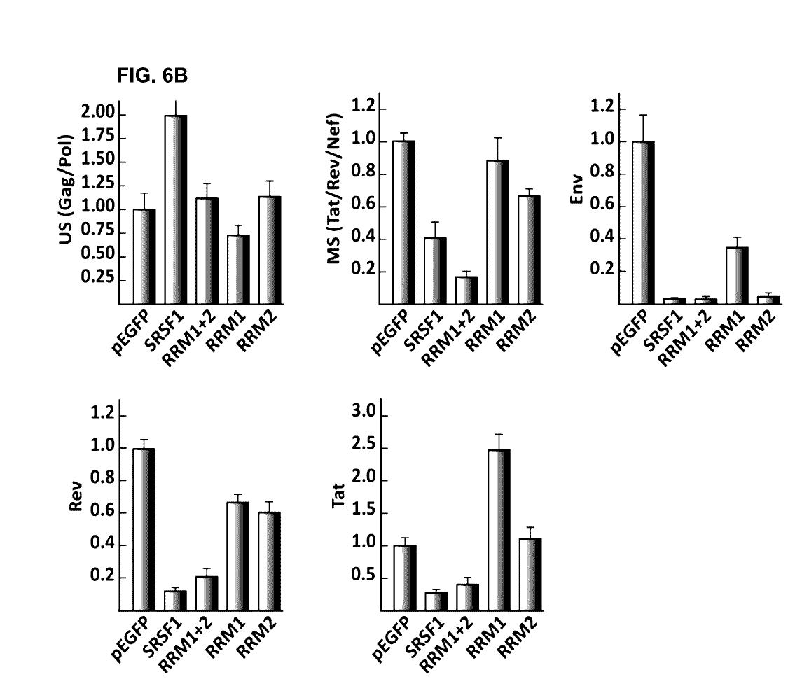

Next, the effect that the SRSF1 mutants have on the levels of viral mRNA and on the splicing of specific viral mRNA isoforms was determined. Messengers were quantified utilizing a series of qPCR assays with primers sets designed to anneal either to a region common to all viral mRNAs or to specific spliced mRNAs species as previously described (Jablonski J A, Caputi M., 2009, Journal of Virology 83:981-992). The wild type SRSF1 down-regulated total viral RNA production by over 20 fold; comparable results were achieved with the clone carrying the deletion of the RS domain or the RRM2 alone while the RRM1 alone decreased viral RNA levels to roughly one third of the control (FIG. 6(A)). Each SRSF1 mutant caused a deregulation of the relative levels of the single viral mRNAs analyzed (FIG. 6(B)), which differs from the pattern obtained upon overexpression of the wild type protein. This was expected given the diverse mechanisms by which SRSF1 exerts its splicing functions and the multiple SRSF1 binding sites present within the viral genome.

These data indicate that SRSF1 exerts pleiotropic effects on the regulation of the viral genome and that its RBDs can inhibit viral replication by presumably competing with other cellular and viral modulators of RNA biogenesis.

The SRSF1 RBDs inhibit divergent viral isolates.

The primary sequence of HIV-1 is highly variable among single isolates. Multiple viral strains with different geographical distribution have been identified. Based on genetic similarities, the numerous virus strains are classified into types, groups and subtypes or clades. More than 90 percent of HIV-1 infections belong to HIV-1 group M. Within group M there are known to be at least nine genetically distinct subtypes. Subtype B is the most common subtype in Europe, the Americas and Japan, subtype C is predominant in Southern and East Africa and India and subtype D is limited to East and Central Africa.

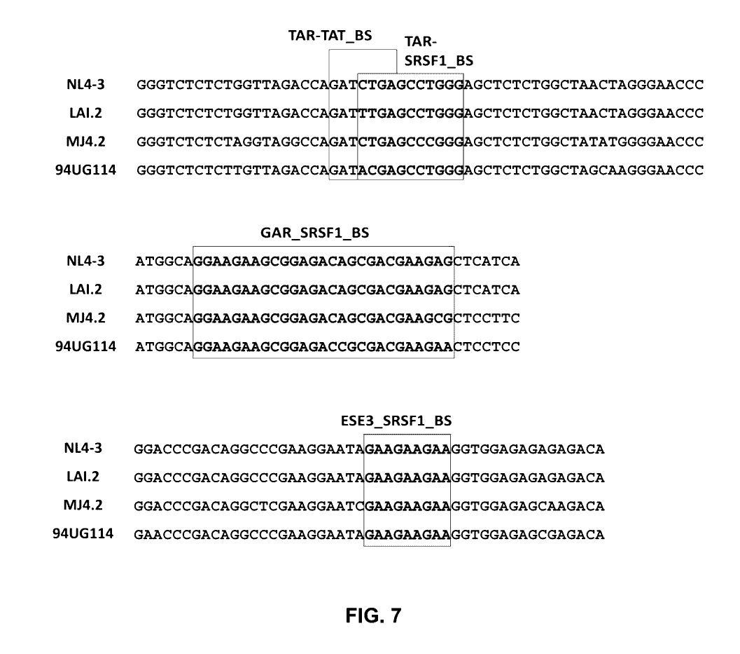

The studies described herein utilize molecular clones derived from early isolates of the B subtype. Since SRSF1 inhibition of HIV replication is dependent on its binding onto multiple sequences within the viral transcript, its activity could greatly vary among different viral strains. Computational analysis of the putative SRSF1 binding sites in the four viral isolates showed multiple potential targets for this cellular factor (FIG. 4(B)). Furthermore, comparison of the binding sites, which have been previously experimentally characterized, revealed a high degree of homology among the viral isolates (FIG. 7).

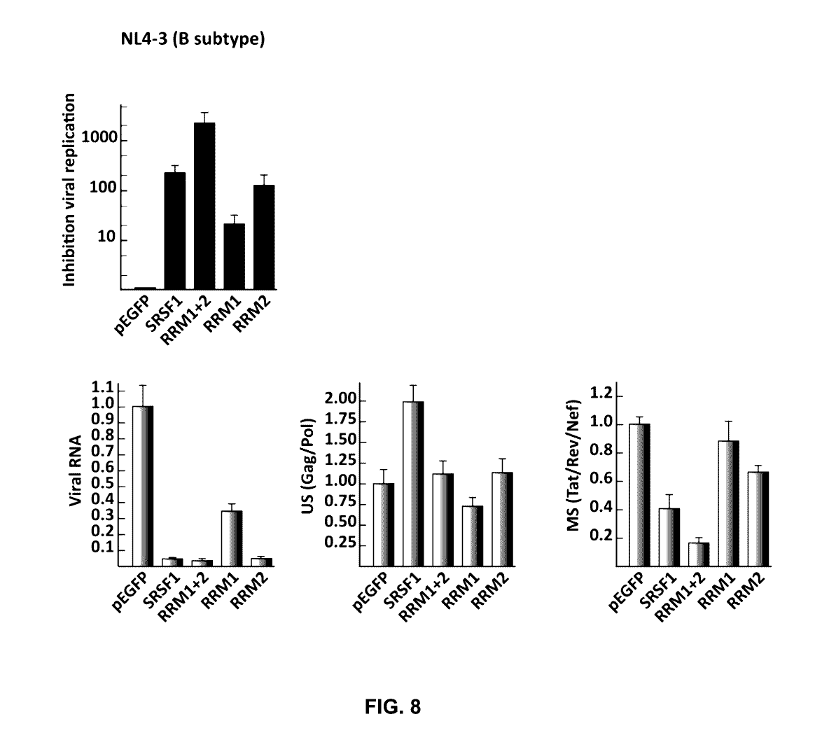

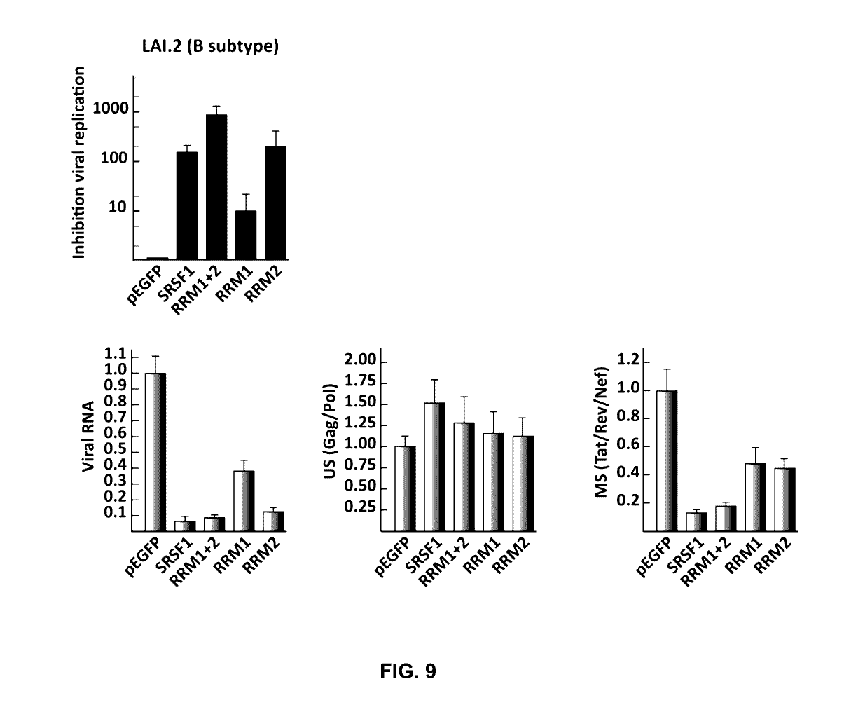

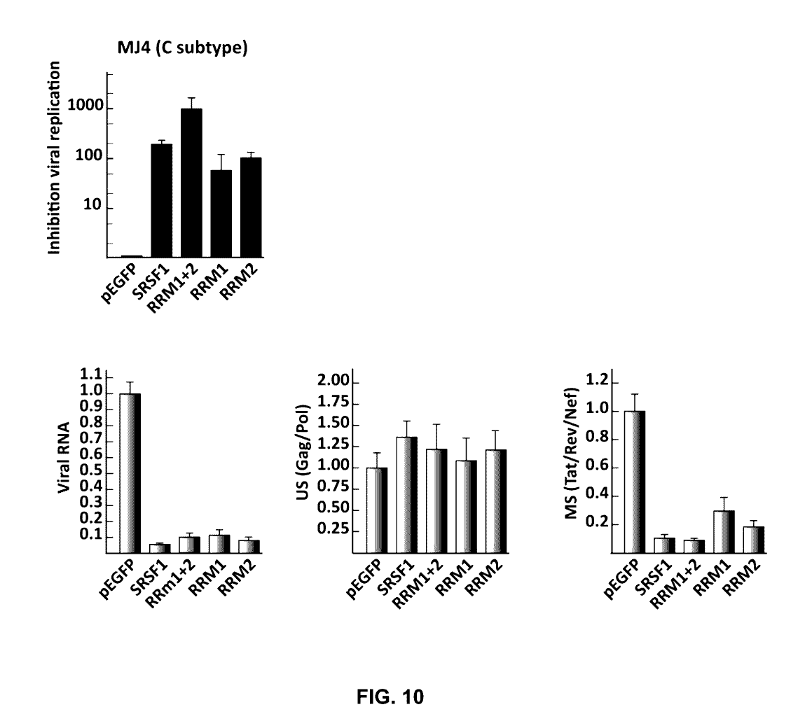

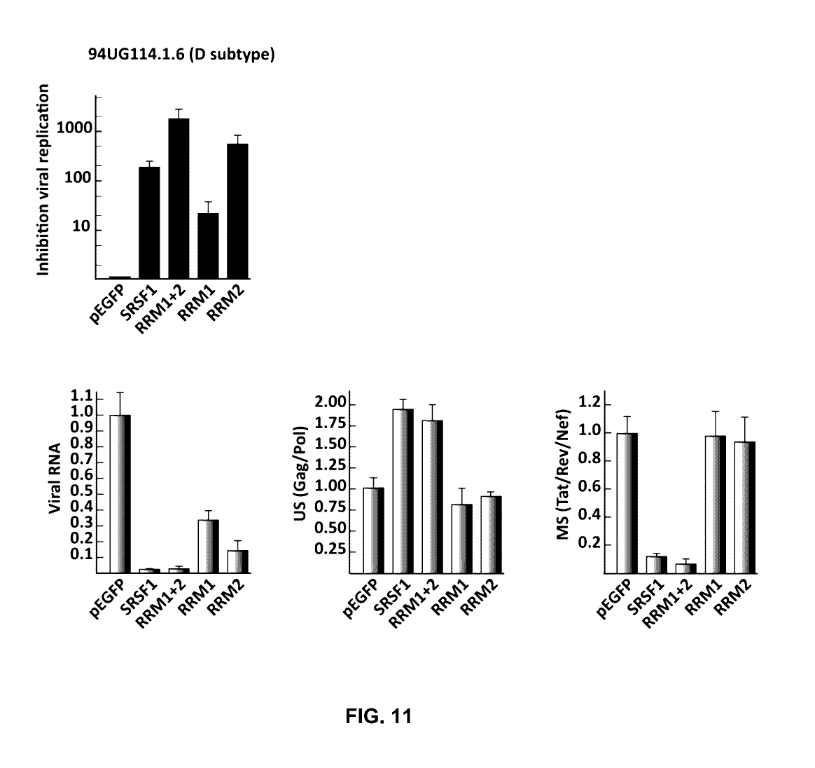

To study the effect of SRSF1 on isolates from the B, C and D subtypes, the production of these viruses in HEK293 cells co-transfected with the molecular clones of each viral isolate and plasmids coding for SRSF1 and its deletion mutants was analyzed. Overexpression of SRSF1 inhibited viral production by at least 100 fold in all the viral isolates (FIG. 8, FIG. 9, FIG. 10, FIG. 11). Consistent with the results obtained with the NL4-3 isolate the strongest inhibitor of viral production was the mutant carrying the RS deletion (over 1,000 fold in all isolates) while the inhibition achieved by the clone expressing the RRM2 alone was comparable to the one achieved by the full length protein. Analysis of the viral transcripts among the different isolates indicated that the intracellular level of all viral messengers were similarly impacted by the SRSF1 wild type sequence and the RS deletion mutant. Expression of the single RRMs lead to more distinctive differences in the relative amounts of unspliced and multiple spliced messengers among the isolates (FIG. 8, FIG. 9, FIG. 10, FIG. 11).

Given the heterogeneity of the sequences recognized by SRSF1 and the different roles played by the single domains in determining its RNA binding specificity, substantial differences in the total and relative amounts of viral mRNAs generated by divergent viruses upon expression of each SRSF1 mutant were expected. Nevertheless, the synthesis and processing of viral mRNAs was severely disrupted in all the molecular clones tested and production of all three viral subtypes analyzed was strongly inhibited by SRSF1 and its single RNA binding domains.

The SRSF1 RBDs have minimal impact on cell viability and apoptosis.

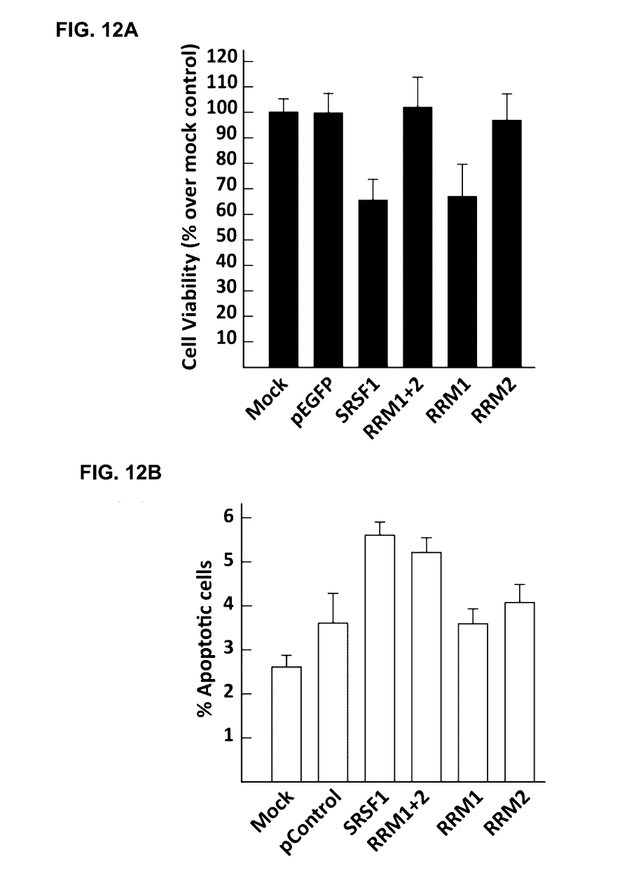

The strong inhibition of viral production observed upon overexpression of SRSF1 suggests a therapeutic utility for this protein. Nevertheless, SRSF1 is an essential gene, which is associated with disease and neoplastic transformation. Thus, it is plausible that its overexpression will induce major changes in cell metabolism, which can result in altered cell viability, proliferation, morphology and apoptosis making it a less likely therapeutic candidate. The data described herein also show that the SRSF1 RRMs can inhibit viral production with an activity comparable (RRM2) or higher (RRMs1+2) to the full-length protein. Given the multiple mechanisms utilized by this protein to exert its cellular functions, it is conceivable that the single RRMs might impact cell metabolism less severely than the wild type sequence. To explore the effects that these deletion mutants have on cell viability and apoptosis, both parameters were measured in HEK293 cells transduced with SRSF1 and its deletion mutants. Cell viability was reduced by over 30% upon overexpression of the wild type SRSF1 and single RRM1 (FIG. 12(A)). Surprisingly, deletion mutants carrying RRM1+2 or the RRM2 alone did not significantly impact cell viability. Apoptotic events increased upon transfection of the wild type and RS domain deletion clones but not the ones expressing the single RRM1 and RRM2 when compared to the control EGFP vector (FIG. 12(B)).

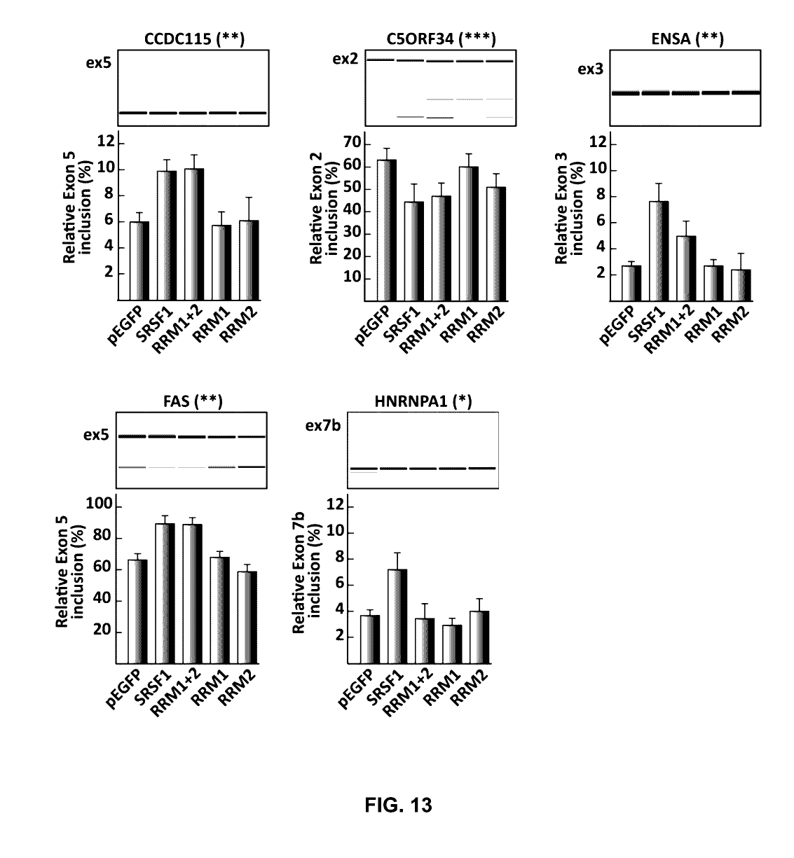

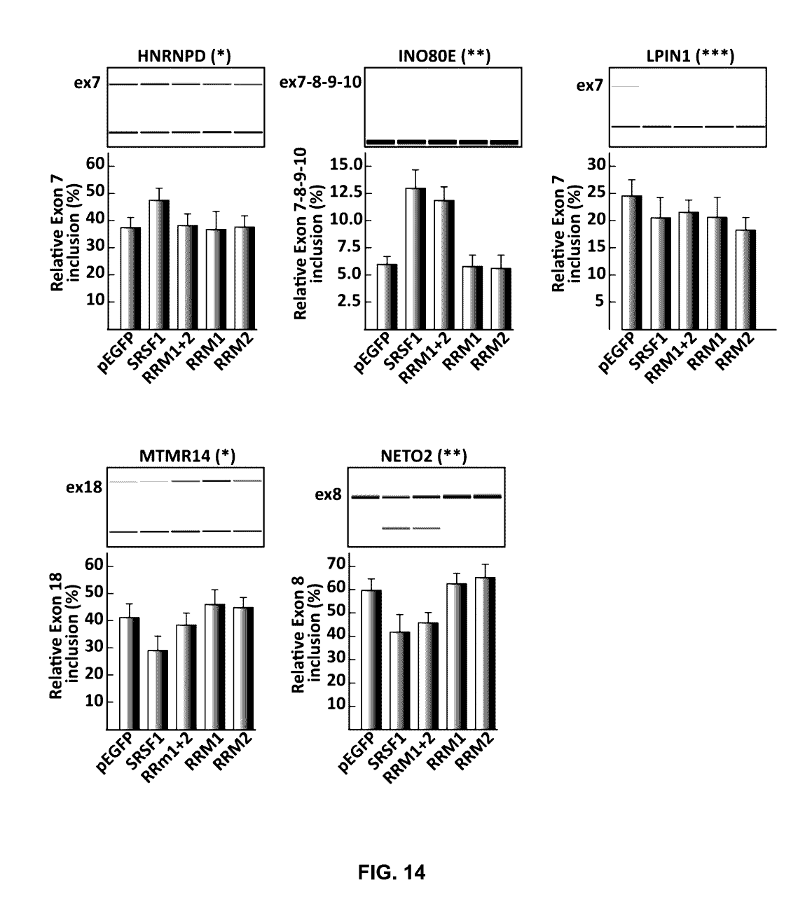

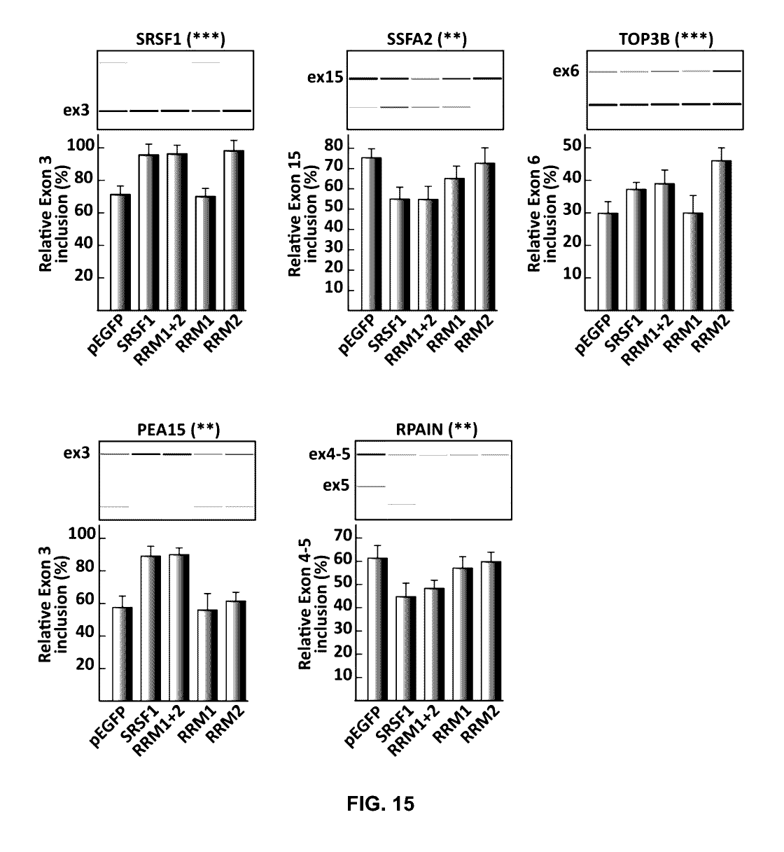

The relevance of this protein's role as a global regulator of transcription, mRNA stability, nuclear export and translation is still unclear. On the contrary, its pervasive role in mRNA splicing regulation is key for cell survival, development and replication. To determine if the deletion clones were differently affecting splicing of cellular genes, a panel of 15 genes, which splicing is regulated by SRSF1 (Clery et al., 2013, Proceedings of the National Academy of Sciences USA 110:E2802-2811; Anczukow et al., 2012, Nature Structural & Molecular Biology 19:220-228), was selected and the inclusion of specific exons was measured. Only 4 of the genes analyzed were similarly regulated by the wild type and the deletion mutants (FIG. 13, FIG. 14, FIG. 15). 3 of the genes surveyed were regulated solely by the wild type SRSF1, while 8 genes were regulated by both the wild type and RS deletion mutant. These data indicate that the SRSF1 RRM domains can inhibit viral replication with a reduced impact on cellular metabolism.

SRSF1 Effects on Viral Replication, Cell Viability and Apoptosis are Dose Dependent.

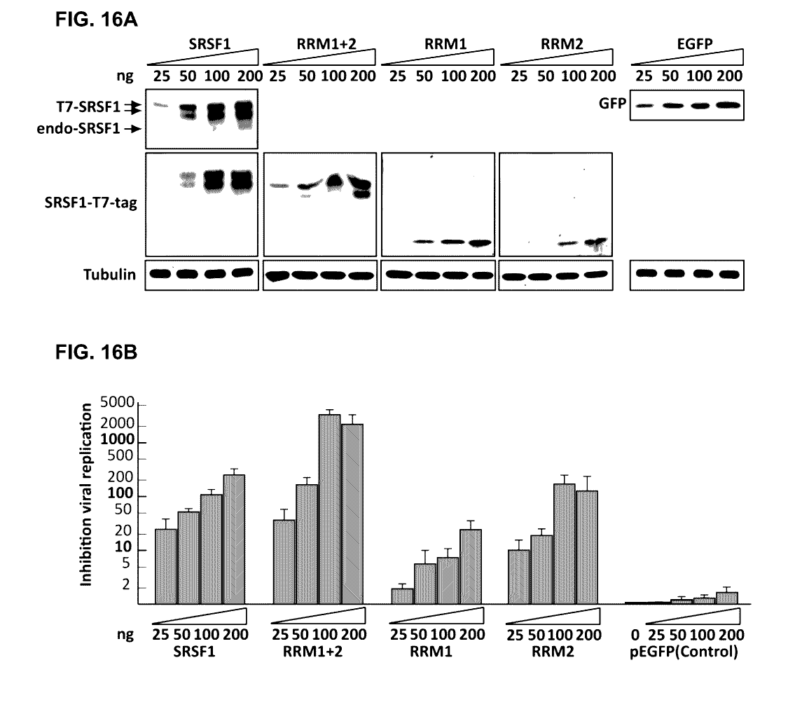

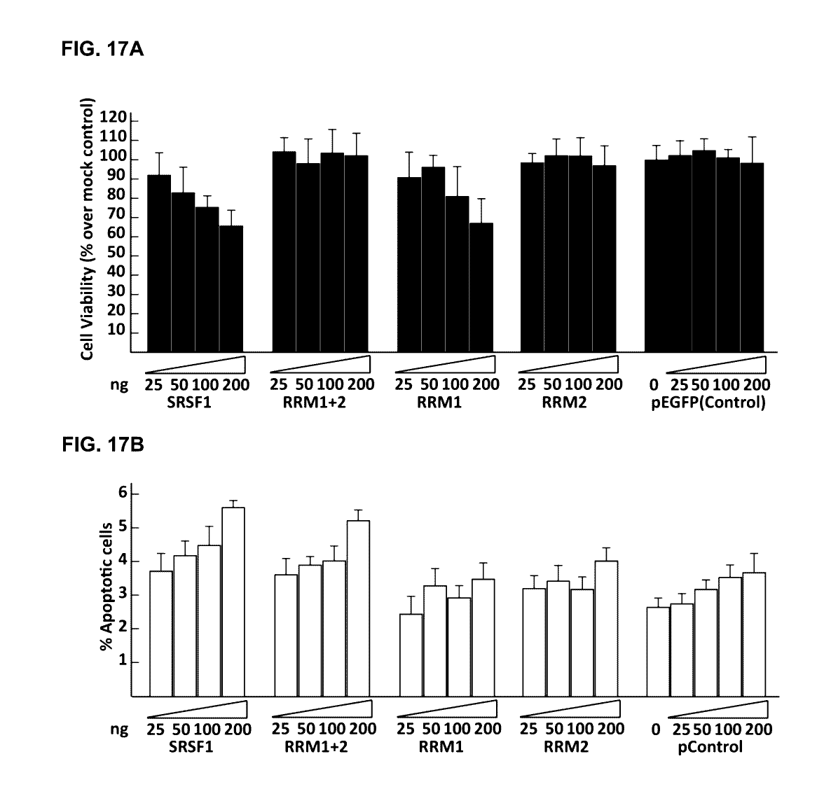

Next, what was the minimal amount of wild type or truncated mutant SRSF1 plasmid that could be transfected to maintain the inhibition of HIV-1 replication and minimize the effects on cellular viability and apoptosis was investigateed. The dose dependent response on the inhibition of HIV-1 virion production (FIG. 16(B)), cell viability (FIG. 17(A)) and apoptotic events (FIG. 17(B)) upon transfection of increasing amounts of the SRSF1 plasmids were analyzed. A reduction by 50% of the input plasmids (FIG. 16(A)) abolished the effects on cell viability and apoptosis for the RS deletion and single RRM2 clones, while maintaining the maximum inhibition of viral production. Cell viability and apoptosis were reduced to control levels when the input of the plasmid coding for the wild type SRSF1 was decreased by 8 fold, this reduced the inhibition of viral production to roughly 20 fold compared to the over 200 fold reduction observed at the higher DNA input.

SRSF1 Inhibits HIV Replication in an In-Vitro Infection System.

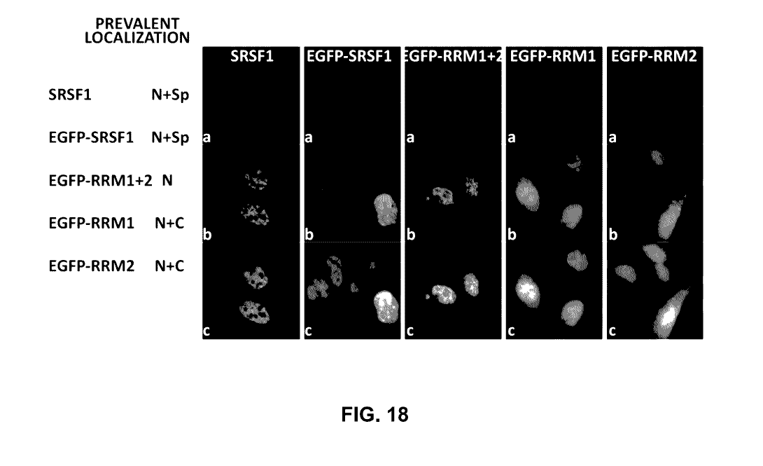

To show that SRSF1 can inhibit viral replication in infected cells that carry an integrated copy of the viral genome, the leukocyte derived H9 cell line, which can be easily infected with a number of viral strains and allows for efficient viral replication, was utilized. H9 cells were infected with viral particles (NL4-3 clone) at high MOI (≧100) and grown for 4 days to ensure the homogenous infection of the cell population. The infected cells were transiently transfected utilizing EGFP tagged SRSF1 clones. The EGFP tagged proteins exhibited cellular localization similar to the untagged ones (FIG. 18). Fluorescence activated cell sorting (FACS) was utilized to select EGFP expressing cells.

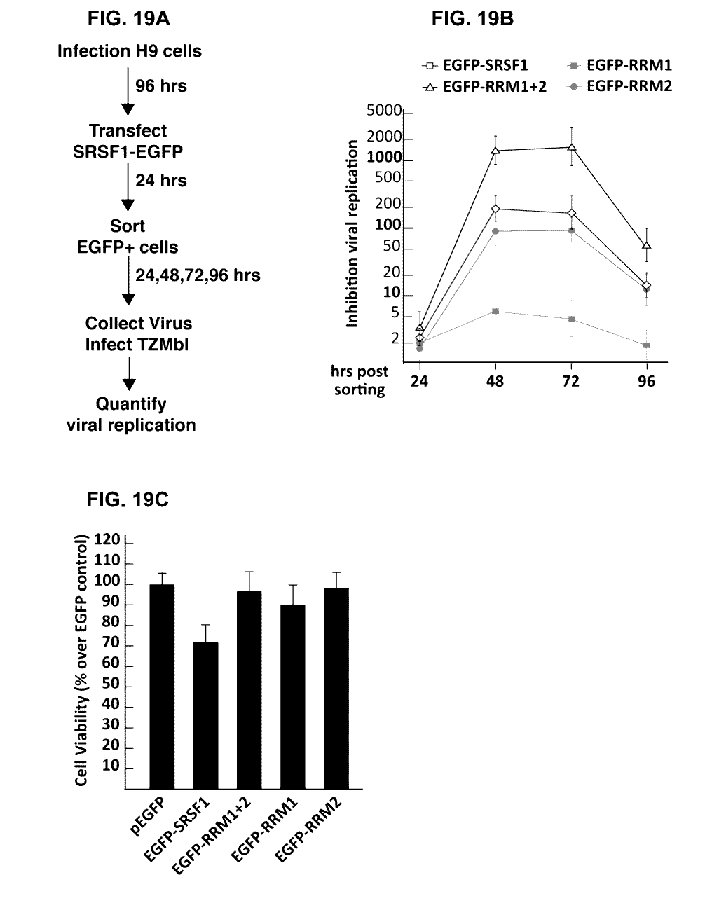

Post-sorting the virus containing media from the infected cells expressing the SRSF1 and control EGFP proteins was collected at 24 hours intervals and utilized to quantify viral production. 48 hours after transfection of the SRSF1 expression clones viral replication was reduced to the levels observed in the viral production assay carried out in HEK293 cells (FIG. 19(B)). At day 4 post-sorting the inhibitory effect of the transfected SRSF1 clones was considerably diminished, this is likely explained by the progressive loss of the epigenic expression plasmid.

Next, the viability of EGFP control and SRSF1 transfected but not infected H9 cells at day 5 post-transfection was examined. Similar to the results obtained in HEK293, the wild type SRSF1 sequence induced a reduction in cell viability while the deletion clones did not (FIG. 19(C)). These data show that viral replication can be inhibited efficiently by expressing the truncated SRSF1 mutants carrying the RRM2 domain either alone (200 fold inhibition) or in combination with the RRM1 domain (>2,000 fold inhibition) without significantly impact cell viability in a stable cell line, suggesting a therapeutic utility for these RBDs.

### Example 2

### Development of Novel Therapies for AIDS

The cellular machineries regulating the transcription and processing of eukaryotic RNAs are intimately coupled. Trans-acting factors required for capping, splicing and polyadenylation are found within the RNA Polymerase II (RNAPII) complex, modulate the processing of the nascent pre-mRNA and, in some cases, processing of the messenger feeds back to regulate the activity of the transcription complex. RBPs have been shown to regulate both transcriptional and post-transcriptional events by connecting the transcription complex to the nascent RNA. In the past years little effort has been aimed at the characterization of the mechanisms connecting viral transcription and RNA processing in HIV. An expression library was utilized to study the role of RBPs in the transcription of the HIV genome and several novel proteins that modulate the activity of the viral transcription complex have been identified. A subset of the proteins identified also regulates the processing of the viral messengers.

The mechanisms by which one of those factors, SRSF1, inhibits the activity of the viral transcriptional transactivator Tat (1) was identified. Binding of SRSF1 to the trans-activating response element (TAR), within the nascent transcript, blocks Tat from entering the active transcription complex. Additionally, it was showed that SRSF1 regulates the splicing of the viral mRNAs by binding to several short RNA sequences. The activity in both viral transcription and splicing of SRSF1 is solely dependent on the protein's ability to bind the target sequence. Expression of the SRSF1 RBDs can strongly inhibit viral replication in model cellular systems (Example 1).

Herein, delivery of the purified SRSF1 RBDs in primary T lymphocytes to inhibit viral replication was investigated. The delivery of a gene expression cassette in leucocytes is primarily carried out by either a lentiviral expression system or by electroporating a plasmid expressing the gene of choice. Both methodologies have several drawbacks: i) electroporation severely damages the cells reducing their viability; ii) the efficiency of delivery for both systems is rarely over 80%; iii) both systems are not ideal to transduce cells in human subjects and at the moment can't be considered for final therapeutic development. To overcome these limitations, purified chimeric proteins containing the SRSF1 RBDs and a cell penetrating peptide (CPP) to deliver the cargo protein to infected T lymphocytes are proposed. A novel type of CPP, &Tat, which has shown enhanced intracellular delivery of the cargo protein with little or no toxicity was utilized and examined.

Described herein is a new platform to study ex-vivo, in physiologically relevant contexts, the role of many cellular factors that have been shown to regulate viral replication in reduced model systems by. Together with their therapeutic potential the reagents generated by this work are likely to yield a better understanding of the role of SRSF1 and other cellular factors in viral infection and latency models.

SRSF1 Inhibits Tat Transactivation.

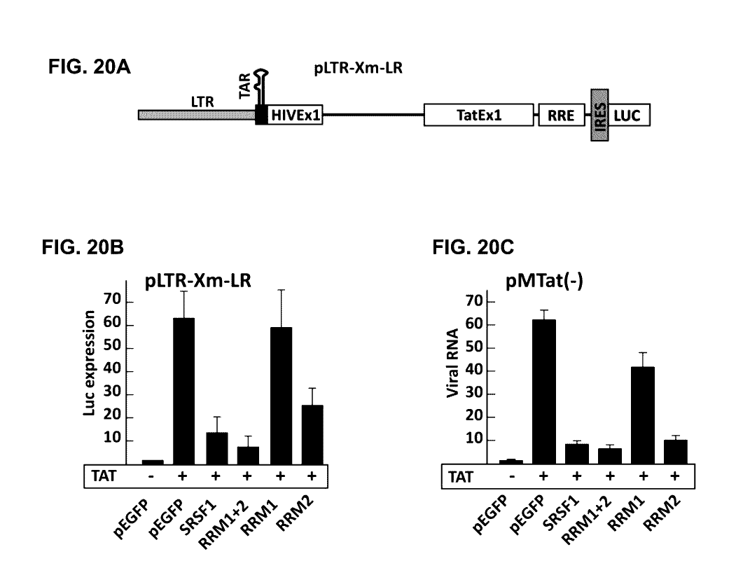

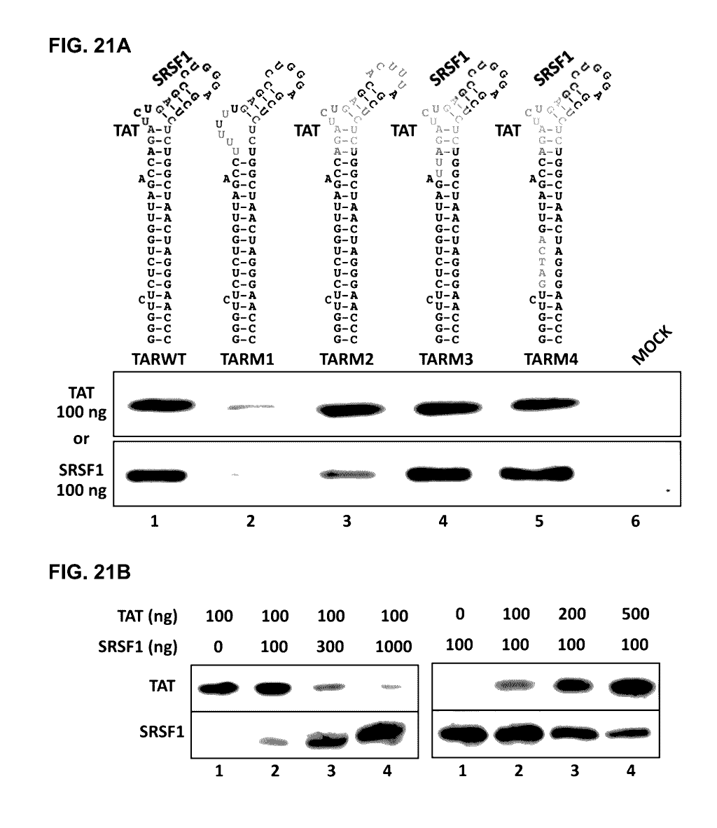

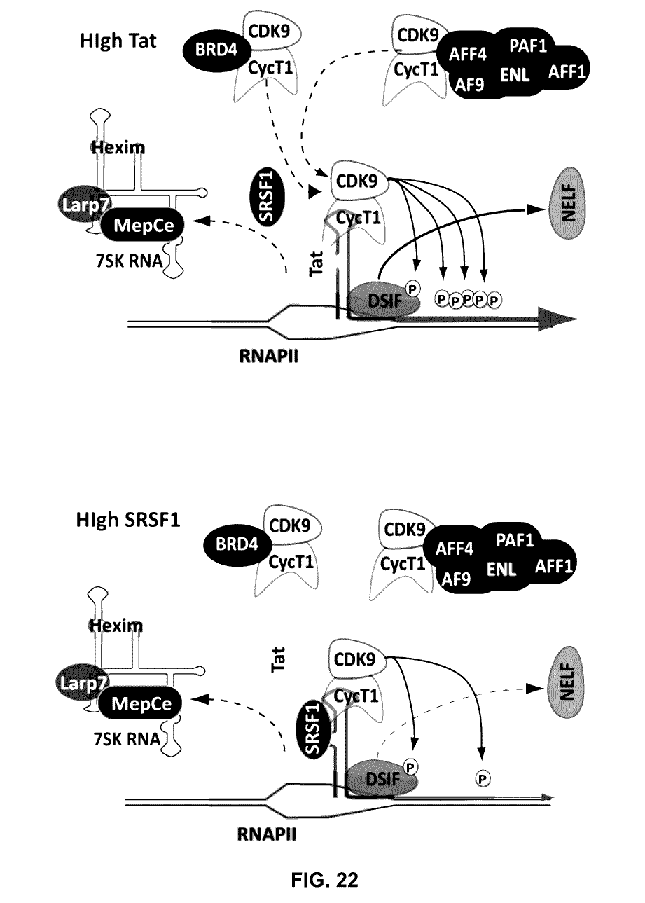

A viral reporter minigenes was utilized to screen a human RBPs expression library for cellular factors regulating viral transcription (1). The activity of cellular factors either up-regulating or down-regulating viral transactivation was confirmed utilizing a full length viral cloning lacking Tat expression pMTat(−) in a transient transfection assay and a cell line carrying the same Tat(−) integrated viral clone. The strongest inhibitor of viral transactivation was the cellular protein SRSF1. Over-expression of SRSF1 inhibited the Tat mediated activation of viral transcription in both the transient expression system and the stable cell line carrying the Tat(−) provirus (FIG. 20(B)). Affinity chromatography and mutagenesis assays showed that SRSF1 and Tat compete for overlapping binding sites onto TAR and their relative concentrations modulate transactivation (1) (FIGS. 21(A), 21(B)). It was also observed that Tat association with the viral promoter decreased upon expression of SRSF1, so did the pTEFb components cyclin T1 and CDK9, which resulted in a decrease of the hyperphosphorylated, transcriptionally active, form of RNAPoIII (1) (FIG. 22).

SRSF1 is composed of two RNA-binding domains of the RRM (RNA Recognition Motifs) type and an RS (arginine/serine-rich) carboxy-terminal domain, which is required for protein-protein interaction but does not appear to affect the RNA binding specificity of the protein (23). Utilizing SRSF1 deletion mutants, it was determined that the protein's inhibition of transactivation is solely dependent on its binding to TAR (1) (FIGS. 21(A), 21(B)) and that RRM2 but not RRM1 was sufficient for the inhibition of transactivation (FIG. 20(B)) (1, 2).

SRSF1 Regulates Viral Splicing.

SRSF1 exerts a key role in the regulation of viral splicing. Several sequences within the viral transcript that are recognized by SRSF1 and regulate the splicing of the viral pre-mRNA by modulating the selection of specific splice sites were characterized (21, 22, 24-26). Furthermore, in-silico analysis of viral sequences from divergent viral isolates indicates that several uncharacterized high-affinity SRSF1 binding sites are preset throughout the viral genome, revealing a pervasive role for this splicing factor in the regulation of the viral pre-mRNA (FIG. 4(B)). Over-expression of SRSF1 or of its RRMs impacts the production of messengers for key genes such a Tat, Rev, and Env. Thus, less Tat is produced in cells expressing high levels of SRSF1 and transcription is further reduced.

To uncouple SRSF1 transcriptional and splicing activities, a viral clone lacking Tat expression, thus lacking transactivation and displaying only basal transcription (FIGS. 1(A), 3(A)), was used. Furthermore, in the analysis of replication competent viruses, the amount of each single mRNA species was normalized for the total viral mRNA content of each sample (Example 1) (FIG. 6(B)). Two mechanisms have been proposed to explain SRSF1 role in viral and cellular splicing regulation (23). In the first SRSF1 recruits, via protein-protein interactions with the RS domain, essential component of the basal splicing machinery onto nearby splice sites. In the second SRSF1 activates splicing by competing with and inhibiting the binding of splicing repressors in the proximity of the regulated splice site. Previous studies indicate that both mechanisms are involved in the regulation of HIV splicing (2, 21, 22, 24-26).

Expression of the SRSF1 RRMs Inhibits Viral Replication.

Given the multiple binding sites present throughout the viral genome, the complex alternative splicing pattern required to generate multiple mRNAs coding for the viral gene products and the diverse mechanisms by which this protein exerts its functions in splicing and transcription it is not surprising that deregulation of SRSF1 expression might considerably alter viral replication. Transient transfection (FIG. 5(A)) and viral infection/replication (FIG. 19(A)) systems were utilized to show that viral replication is down-regulated by SRSF1 over-expression.

The primary sequence of HIV-1 is highly variable among isolates. Viruses found within the group M cause more than 90 percent of HIV-1 infections. Within this group there are at least nine genetically distinct subtypes. Subtype B is the most common subtype in Europe, the Americas and Japan, subtype C is predominant in Southern and East Africa and India and subtype D is limited to East and Central Africa. Since SRSF1 functions are dependent on the recognition of multiple sequences within the viral transcript its activity could vary among different viral strains. We analyzed the replication of viral isolates of the B, C and D subtypes in cells over-expressing SRSF1. Viral replication was inhibited by a minimum of 100 fold upon over-expression of SRSF1 in all the viral isolates tested (Example 1) (FIGS. 8, 9, 10, 11).

The strong inhibition of viral replication observed upon over-expression of SRSF1 suggests a therapeutic potential for this protein. Nevertheless, SRSF1 is an essential gene which expression is associated with disease and neoplastic transformation. It is plausible that its expression will induce major changes in cell metabolism, viability and proliferation making it a less likely therapeutic candidate. To overcome these problems we utilized SRSF1 deletion clones expressing the protein RRMs but lacking the RS, protein-protein interaction domain. In Hek293 cells expression of the SRSF1 RRMs had a minimal impact on cell viability and proliferation while inhibiting viral production with an activity comparable (RRM2) or superior (RRM1+2) to the full-length protein (Example 1) (FIGS. 12(A), 12(B)). Comparable data were obtained in an infection assay utilizing H9 cells (FIGS. 19(A), 19(B), 19(C))

To determine the therapeutic potential of SRSF1 it is necessary to determine this protein's role in viral replication in a physiological relevant infection model, this is why much of the work described herein aims at the transduction of the SRSF1 RRM domains in primary CD4++T cells, which are the main viral target.

Lymphocytes are efficiently transduced by the Tat Cell Penetrating Peptide (CPP) conjugated to a protein cargo.

Since SRSF1 RRMs can efficiently inhibit viral gene expression and replication without significantly alter cell viability in stable cell lines our goal is to establish a system to deliver the SRSF1 RRMs in primary T cells to block HIV replication and expand the study of the role of this protein in cellular and viral metabolism. The majority of the approaches utilized to transduce leukocytes are based on lentiviral or electroporation systems. Both methodologies have drawbacks and do not assure the homogenous transduction of the treated cells. Electroporation severely damages the cells and its efficiency is seldom above 60%, while lentiviral delivery systems vary greatly in their efficiency, which is rarely above 80%. Thus, further manipulations are often needed to obtain a homogenous population of transduced cells. Furthermore, both systems are less than ideal when utilized to deliver a gene of choice in-vivo. To overcome these limitations, the use of CPP to deliver the cargo SRSF1 protein to primary T cells is proposed.

Cell penetrating peptides are a group of short cationic sequences with a remarkable capacity for membrane translocation (27). CPPs can be conjugated to large proteins and other macromolecules, cross highly selective barriers, such as the intestinal wall or the blood-brain barrier, and deliver the biologically active cargo. Among others, the HIV-1 Tat-derived peptide (hereafter Tat CPP; sequence: YGRKKRRQRRR (SEQ ID NO:3)) has been shown to successfully deliver a large variety of cargoes, from small particles to proteins and nucleic acids in most cell types (28). The Tat CPP utilizes multiple pathways for cellular entry. When attached to small cargoes can directly translocate across the plasma membrane. However, when conjugated to macromolecules the Tat CPP enters cells using the endocytic pathway (29). Large cargoes, proteins, and peptides can be delivered intracellularly with high efficiency if conjugated to the Tat CPP in difficult to transfect primary cells and animal models. More specifically, lymphocytes in culture and in mice can be efficiently transduced with Tat CPP-conjugated peptides (30).

Inhibition of HIV-1 Replication Utilizing Chimeric TatCPP:SRSF1-RRMs Proteins.

The goal is to inhibit viral replication in human leukocytes, the primary target of the virus, with a chimeric peptide constituted by the SRSF1 RRM1+2 or RRM2 alone coupled to the TatCPP. The fusion proteins are synthesized and purified in a bacterial system. The chimeric proteins are utilized to inhibit viral replication in leukocyte-derived cell lines and CD4++T cells purified from healthy donors.

Cloning and Purification of the TatCPP:GB1:RRMs Chimeras.

The pET (Novagen) system is utilized to express and purify chimeric recombinant proteins in bacteria. The Tat CPP and a His-Tag is inserted at the N-terminus of either the SRSF1 RRM1+2 (residues 1-203 corresponding to SEQ ID NO:1) or the RRM2 alone (residues 107-203 corresponding to SEQ ID NO:1). Furthermore, since a known drawback of bacterial expression systems is that ⅔ of soluble proteins are characterized by low solubility and instability at high concentrations, the protein G B1 domain (GB1, 56 residues) (31) is added to increase the solubility and stability of the chimeric protein. Previous work has shown that addition of the non-cleavable GB1-Tag to the C-terminus of the SRSF1 RRMs does not interfere with their folding and ability to interact with their target RNA while increasing solubility up to 0.8 mM (32).

Although it is difficult to predict the effect of the addition of residues on a protein's functions, the data shown in FIG. 10b shows that addition of the TatCPP, GB1 and His sequences to the C-terminus of RRM1+2 and RRM2 did not alter the antiviral activity of the RRMs. These results give confidence that, upon delivery to the cell, the TatCPP-GB1-RRMs chimeras will retain the antiviral functions of the SRSF1 RRMs.

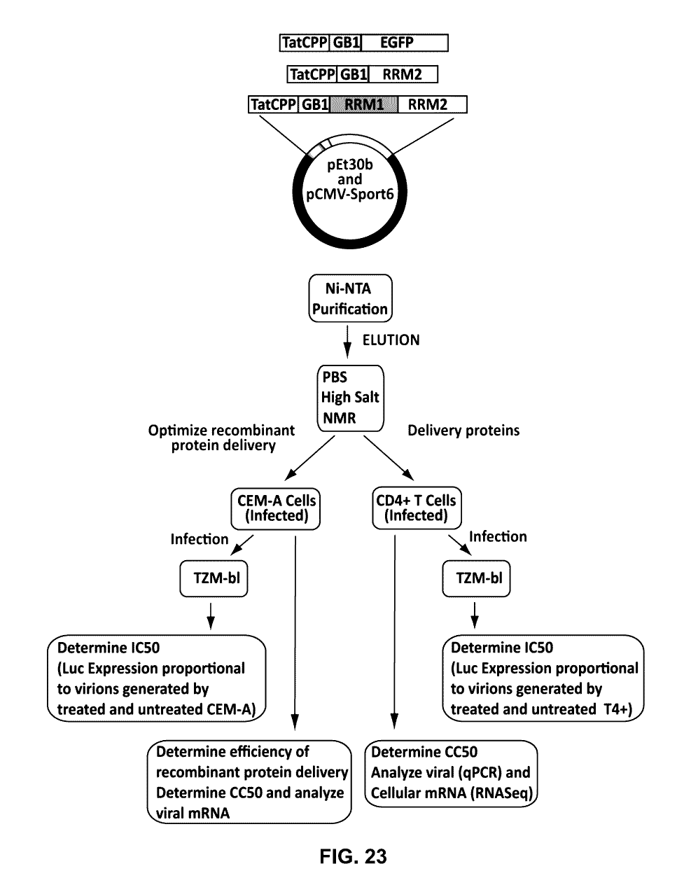

Proteins are purified utilizing a procedure previously described to purify RRMs in higher concentration for NMR and RNA binding studies (32, 33). Proteins are expressed in E. coli, purified by nickel affinity chromatography, eluted in native conditions, dialyzed and concentrated as described (32). Protein is dialyzed and stored in three alternative buffers. Two of the buffers (NMR, and High Salt) have been shown to allow storage of the GB1-tagged SRSF1 at high concentrations (>200 μM) (32), the third, PBS, is widely used to store Tat-CPP fusion proteins in several systems (34, 35). The proteins preparations are tested and compared for their cytotoxicity and ability to inhibit viral replication (FIG. 23).

Regarding solubility of the proteins, if necessary to avoid protein aggregation, a urea denaturation purification protocol followed by rapid desalting can be utilized for over 50 TatCPP-fusion proteins (34). If purification of proteins at a concentration higher than 100 μM is problematic, a commercial service (e.g., Bio-Synthesis), which can efficiently synthesize long peptides, can be used.

Validate and Optimize Delivery of TatCPP:GB1:RRMs Chimeric Proteins in the CEM-A Cell Line.

The recombinant proteins are tested for their delivery efficiency and cytotoxicity in CEM-A cells, a lymphocytes derived cell line, which can be infected by several viral isolates and grows in adhesion (FIG. 23). The chimeric proteins transduction is analyzed by immunohistochemistry. The delivery of each recombinant protein stored in NMR, High Salt or PBS buffers is compared, and the optimal concentration and incubation time for each preparation determined. The protein preparations is tested in triplicate in a 7-point 1:3 dilution series starting at a nominal test concentration of 10 nM. Cytotoxicity is evaluated colorimetrically by measuring mitochondrial dehydrogenase activity in living cells via cleavage of the XTT tetrazolium salt in the presence of phenazine methosulfate. Apoptosis is determined colorimetrically utilizing commercially available kits (Biocolor). The 50% cytotoxic concentration (CC50) is calculated for each protein preparation. Given the low toxicity exhibited by expressing the SRSF1 RRMs (FIG. 12(A)) and the ample data available on the toxicity of the TatCPP-, His- and GB1-tags, the CC50 is expected to be in the high mM range.

Next, the chimeric proteins are evaluated for their capacity to block production of the HIV-1 virus. CEM-A cells are infected with viral preparations at high MOI (=10). Viral stocks are generated by transfecting HEK293 cells with proviral constructs coding for viral molecular clones of the B, C and D subtypes (pNL4-3, pMJ4, p94UG114.1.6). Viral infectious titers are determined in TZM-bl cells as previously described (36). The homogenous population of infected cells are treated with different amounts of the recombinant proteins and supernatants are collected at 24 hours intervals after addition of the proteins and are used to infect the TZM-bl reporter cell line. The half maximal inhibitory concentration (IC50) is calculated by measuring luciferase expression in the reporter TZM-bl cells. Viral transcription and splicing in the treated and untreated infected CEM-A cells are quantified by utilizing the qPCR methodology previously described (9).

The activity and toxicity of the proteins in each of the three storage buffers utilized (NMR, High Salt or PBS) are compared. Preparations with the higher therapeutic index (TI) given by the CC50/IC50 ratio are selected for the inhibition of virion production in CD4++T cells as described below. If the observed CC50 to IC50 ratio is under 100, a different CPP tag can be utilized. The antenopodia (AntpHD) (37) and the PEP-1 (38) are widely used in cellular and animal studies while the HHph-1-PTD, has been successfully utilized to deliver a fusion protein in human T cells (39). If a large portion of the TatCPP:GB1:RRM1 and 2 is retained in the endosomes, endosomal release can be improved by adding a short endosomolytic peptide derived from the influenza virus hemagglutinin-2 (HA2) (40) to the chimeric protein.

Inhibition of Virion Production in CD4+ T Lymphocytes.

Whether or not delivery of TatCPP:GB1:RRMS1+2 and TatCPP:GB1:RRMS2 reduces viral replication in CD4+ T lymphocytes, the primary target of the virus, is determined. Whole blood leukapheresis from a healthy donor is obtained from OneBlood a non-for profit, Florida based, blood center. Peripheral blood mononuclear cells (PBMCs) are purified by Ficoll-Paque density centrifugation. CD4+ T lymphocytes are isolated from PBMCs by negative selection (LifeTechnologies, Untouched™ Human CD4 T Cells Kit) and activated by incubation with IL2 and anti-CD3/28 antibodies. Following activation CD4+ T lymphocytes are infected with the viral stocks as described above. The infected cells are treated with the recombinant proteins preparation (NMR, High Salt or PBS buffers) with the higher therapeutic index (CC50/IC50). The internalization of the recombinant proteins is analyzed by immunohistochemistry and quantified by western blot. Inhibition of viral replication is carried out utilizing the reporter cell line TZM-bl as described above at 24 hours intervals. The inhibition of viral transcription and splicing is quantified by qPCR (9). The IC50 and CC50 of the chimeric proteins are calculated in CD4++T cells.

To study the off target effects caused by the delivery of the fusion proteins, global changes in the transcriptome of the treated CD4++T cells are analyzed by RNASeq. The RNASeq data analysis provides genome wide information on genes that may be regulated transcriptionally or post-transcriptionally by delivery of the TatCPP:GB1:RRMS1+2 or TatCPP:GB1:RRMS2. Preliminary RNASeq assays carried out in HEK293 cells show a small subset of cellular genes (>50) which expression is altered shortly (12 hours) after SRSF1 over-expression and a larger number (>500) displaying an altered splicing pattern.

The recombinant proteins are expected to have a therapeutic index (CC50/IC50) of at least 100-fold above the IC50 value. If delivery of a sufficient amount of the recombinant protein to the CD4++T cells to efficiently inhibit viral replication is problematic, a lentiviral system approach can be used to deliver an expression cassette coding for the SRSF1 RRM1+2, RRM2 or EGFP to CD4++T cells. Since lentiviral transduction efficiency in T cells is often lower than 90%, a lentiviral system (System Bioscience) which transduces a fluorescent marker together with the target gene may be used. The fluorescent marker is utilized to select a homogeneous population of transduced cells.

Delivery of Chimeric Proteins with Increased Specificity for HIV-1 Infected Lymphocytes.

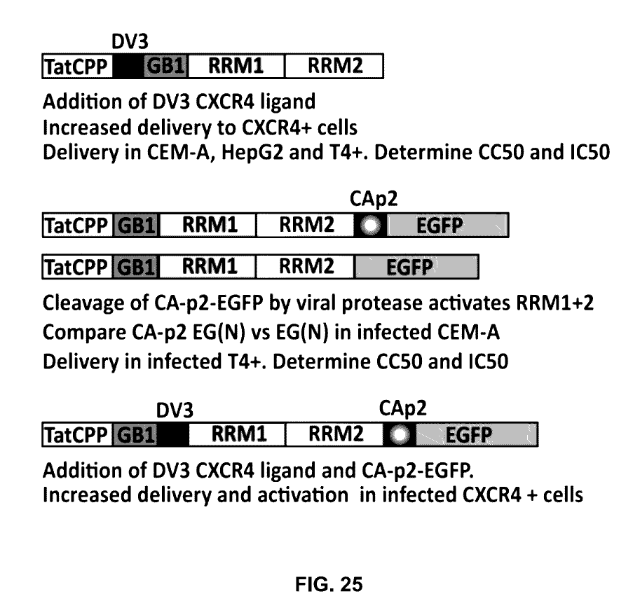

A drawback in utilizing the Tat CPP is its ability to efficiently transduce any cell type. To improve the cell specificity and limit the activity of the TatCPP:GB1:RRM1+2 protein to HIV-1 infected lymphocytes, a cell-type specific homing peptide is used, and the chimeric protein is modified to render it functionally active only in infected cells (summarized in FIG. 25).

HIV-1 recognizes and infects cells expressing the CXC chemokine receptor 4 (CXCR4). DV3 is a short CXCR4 ligand (LGASWHRPDKG (SEQ ID NO:14)), which has been shown to specifically target CXCR4 expressing cells and increase the internalization of a cargo protein when linked to a chimeric protein containing the Tat CPP (41). The DV3 sequence is added to the TatCPP:GB1:RRM1+2 protein to improve the cell-type specificity of the fusion protein. Proteins are purified as described above. The internalization of the chimeric proteins containing or lacking the DV3 peptide in cells, which either do (CEM-A) or do not (HepG2) express the CXCR4 receptor, is compared by imaging and western blot. The IC50, CC50 and therapeutic index values for the DV3 containing chimeric proteins are determined. The inhibition of virion production by the DV3-containing chimeras in CD4+ T lymphocytes is determined.

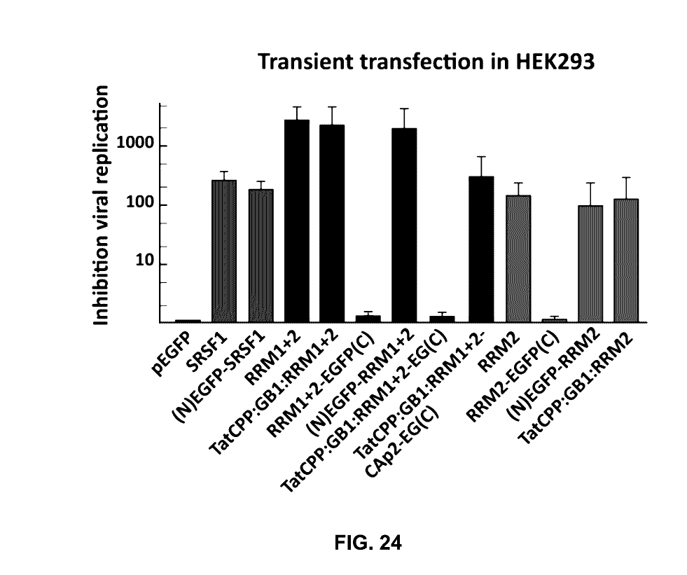

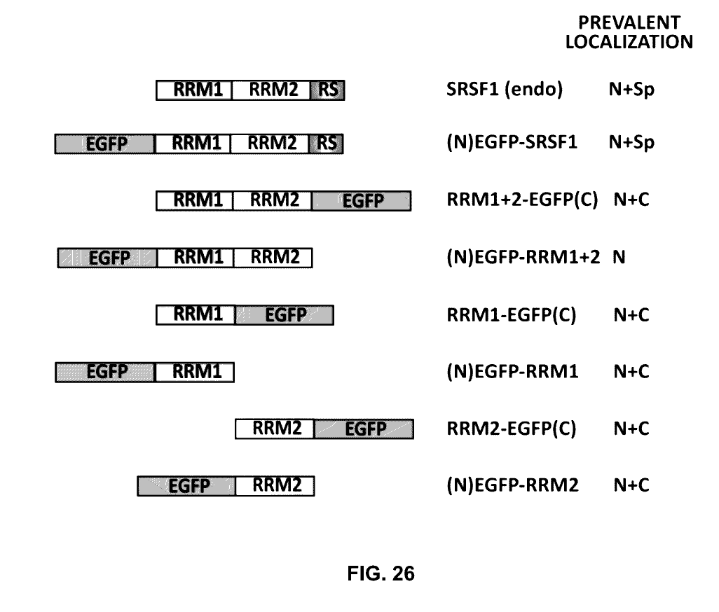

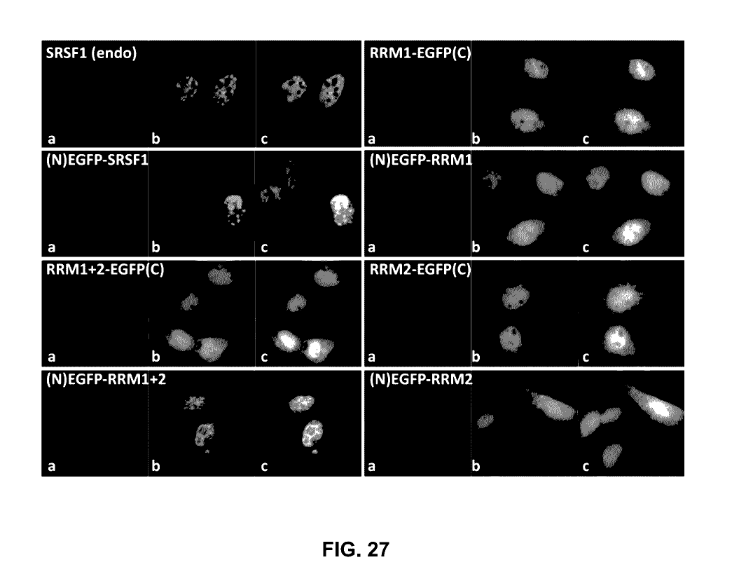

To limit the specificity of the TatCPP:RRM1+2 chimeric protein to infected cells, a fusion protein with the ability to enter cells in a pro-drug manner is engineered. The approach previously utilized to kill HIV infected cells using the Tat CPP linked to a ProCaspase-3 (Casp3), where its endogenous cleavage sites were substituted with HIV proteolytic cleavage sites, is used, so that the proteins can be cleaved into its active form only in cells expressing the HIV protease, resulting in apoptosis of the infected cells. (42). The data shown in FIG. 24, FIG. 26 and FIG. 27 show that addition of the EGFP protein to the N-terminus of the SRSF1 RRM1-2 inhibits the antiviral activity of the protein subdomains. A bacterial expression construct containing the EGFP coding sequence added to the N-terminus of the TatCPP:GB1:RRM1+2 chimera is engineered and a consensus HIV proteolytic cleavage site is inserted between the RRM1+2 and EGFP coding sequences (FIG. 24). Cleavage of the EGFP sequence from the fusion protein by the viral protease is expected to induce the antiviral activity of the RRM1+2. The data shown in FIG. 24 indicate that addition of the CA-p2 cleavage site upstream the N terminal EGFP tag restores the antiviral activity of the RRM1+2:EGFP(C) chimera, thus validating this approach. The TatCPP:GB1:RRM1+2:EGFP(C) chimeras containing or lacking the HIV CA-p2 cleavage domain (ARVLAEAM) between the RRM1+2 and EGFP sequences (FIG. 25) are purified as described above.

The antiviral activity of the TatCPP:GB1:RRM1+2:EGFP(C) chimeras containing or lacking the HIV CA-p2 cleavage domain in HIV-1 infected CEM-A is validated. The inhibition of viral replication by the TatCPP:GB1:RRM1+2:Ca-P2:EGFP(C) chimeras in CD4+ T lymphocytes is determined. If both approaches are successful, a new construct containing both the DV3 peptide and the Ca-P2:EGFP tags is created to further increase the specificity of the chimeric protein to infected cells. The new chimeric protein is purified and tested in CEM-A and primary T4+ cells as specified above.

The ability of the TatCPP tagged proteins to penetrate and be active in any cell type makes them the ideal tool to investigate the role and therapeutic potential of proteins such as SRSF1 in metabolically inactive and hard to study cells, which constitute the majority of the latent viral reservoir.

Study and Optimize the Sequences Required for Optimal HIV-1 Inhibition.

SRSF1 antiviral activity is the result of the protein's affinity for specific RNA sequences and the inhibition of specific nuclear events: the transcription and processing of the viral messenger. The RNA-binding specificity of SRSF1 appears to be mostly dependent on RRM2, since it interacts with the target RNA with efficiency similar to the one of RRM1 and 2 combined (32). The data described herein indicate that inhibition of viral replication differs greatly upon expression of RRM2 and RRM1+2 (˜200 fold inhibition vs ˜2000 fold inhibition), thus factors other than the RNA binding specificity of these subdomains may account for the inhibition of viral replication. SRSF1 is a shuttling protein primarily localized within the nucleus and associated with spliceosomal components in subnuclear structures named nuclear speckles (43). Deletion of the SRSF1 RS domain (RRM1+2 mutant) has little impact on the prevalent nuclear localization of the protein while the single RRM1 or 2 domains show a diffuse cellular localization (44) (FIG. 26, FIG. 27). Thus suggesting that the different efficiency in antiviral activity observed upon expression of the RRM1+2 compared to the RRM2 alone is due to their intracellular localization (nuclear, associated with speckles vs diffused). Since a minimal NLS (GRKKR) (SEQ ID NO:15) is also contained within the Tat CPP it is possible that the proteins will localize within the nucleus in the absence of other specific NLS, although this does not appear to be the case in most Tat CPP fusion proteins. To increase the intracellular localization of the RRM2 recombinant proteins, the minimal NLS signal present within RRM1+2 is determined. Furthermore, the precise SRSF1 sequences required for HIV transcriptional and splicing inhibition are determined.

Determining the RRM1-2 NLS and Other Key Sequences.

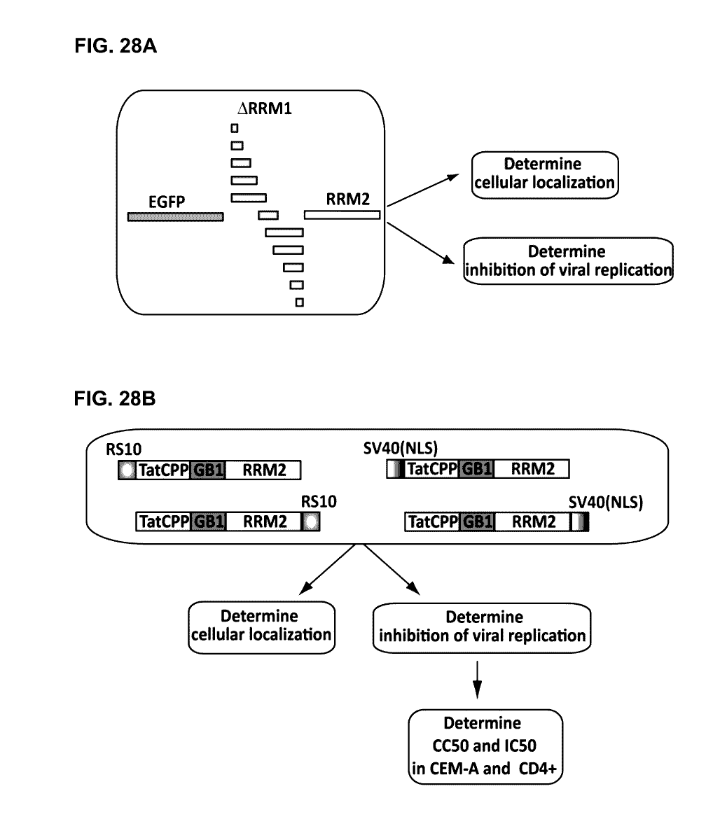

The sequence that allows for the prevalent nuclear localization of RRM1+2 is determined by analyzing a library of deletion mutants. Different fragments derived from the RRM1 are added between the EGFP and the RRM2 in pEG-RRM2 to obtain a series of pEG-ΔRRM1+2 clones, the minimal sequence required for efficient nuclear localization is determined by confocal microscopy (FIG. 28(A)). The minimal pEG-ΔRRM1+2 clone showing subcellular distribution similar to pEG-RRM1+2 are tested for its ability to down-regulate viral replication in the transient transfection system summarized in FIG. 5(A). Briefly, HEK293 cells are co-transfected with the proviral vector pNL4-3 and the pEG-ΔRRM1+2 clones. Viral replication is quantified by infecting the reporter cell line TZM-bl with the supernatant collected from the transfected HEK293 cells. If the minimal deletion clone shows an increase in antiviral activity when compared to the RRM2 alone, the minimal sequence ΔRRM1+2 is conjugated with the Tat CPP and the antiviral activity of the purified proteins compared with one of the NLS:RRM2 chimeric proteins described below.

To determine the precise SRSF1 sequences required for inhibition of transactivation and viral replication, a targeted mutagenesis approach is used. The minimal sequence required for nuclear localization as determined above is mutagenized within the RRM1+2 sequence and tested following the process schematized in FIG. 23. Next, key nucleotides within RRM1 and RRM2 that have been previously shown to be required for their RNA binding functions and other subdomains residues shown to modulate SRSF1 activity in other systems (32, 33) are mutagenized.

Cloning, purification, validation and delivery of a NLS:RRM2 chimeric protein. To increase the nuclear localization and antiviral activity of the SRSF1 RRM2, the activity of two different short NLS sequences: i) the RS decarepeat (RS)10 which is sufficient to confer SRSF1 the proper subnuclear localization into speckles (45) and ii) the canonical short NLS from the simian virus 40 (SV40) large T antigen (PKKKRKV) (SEQ ID NO:16), which has been shown to confer nuclear localization to several proteins (46), are compared.

Expression clones (bacterial and eukaryotic) containing either the SV40 NLS or the (RS)10 peptide joined to the TatCPP:GB1:RRM2 coding sequence (FIG. 28(B)) are compared. Proteins are purified as described above. The antiviral activity and cellular localization of the fusion proteins are tested in a transient transfection system as described above.

Recombinant proteins are purified and the antiviral activity and toxicity of the NLS-tagged and untagged proteins are compared. The internalization efficiency, intracellular localization and cytotoxicity of the purified proteins in CEM-A cells is determined and the inhibition of virion production in CEM-A cells is analyzed. The NLS containing chimeric protein with the higher therapeutic index, determined in CEM-A cells, is used to inhibit viral replication in purified CD4++T cells as described above (FIG. 23). The inhibition of viral replication is monitored utilizing the reporter cell line TZM-bl and the therapeutic index for each chimeric protein in CD4++T cells is calculated (FIG. 28(B)).

Initially the NLS sequences are positioned at the N-terminus of the chimeric protein. Nevertheless, a more efficient nuclear localization might be achieved by inserting those sequences in a different position within the chimeric protein. If no- or only marginal-increase in the nuclear localization of the fusion proteins is observed upon addition of the SV40 and RS(10) NLS at the N-terminus of the construct, the NLS is inserted at the C-terminus of the construct. Furthermore, other short NLS sequences, such as the Epstein-Barr virus DNase NLS (47) and the bipartite human hnRNP K NLS (48), can be utilized. A minimal recombinant protein, ideally less than <150 aa, with optimal antiviral activity and minimal cytotoxicity, which can be easily synthesized utilizing conventional biochemical systems, is generated. Larger amounts of this biologically active long peptide can be synthesized by commercial providers. Chemical synthesis of the peptides allows for better quality control and higher purity than what may be obtained utilizing a bacterial expression system.

Delivery of the SRSF1 RRMs to Infected Cells Utilizing the Endosomolytic Agent &Tat.

A novel strategy to deliver peptides to live cells, which combines efficient endosomal escape, low cell toxicity and convenience has been recently developed by Dr. Pellois (52). A dimer of the cell-penetrating peptide Tat labeled with the fluorophore tetramethylrhodamine (TMR), named dfTat (dimeric, fluorescent Tat), has been shown to penetrate cells by escaping from endosomes with high efficiency and deliver protein cargoes into cells after a simple co-incubation procedure. dfTAT can deliver proteins into cells in ˜1 hour without affecting cell viability or proliferation. Delivery does not require covalent binding interactions between dfTat and the cargo protein and can be repeated several times.

The intracellular concentration of SRSF1 in HEK293 (˜0.5 μM) is comparable to the one found in T cells (˜0.3 μM). The data described in Example 1 indicate that expression of SRSF1 RRM1+2 to roughly 10 fold the concentration of the endogenous SRSF1 decreases viral replication by over 2000 fold (2). Thus, in CD4++T cells a RRM1+2 intracellular concentration >5 μM should be sufficient to achieve a substantial reduction in viral replication (>log₁₀3). Incubation of several primary cells with dfTat (5 μM) and EGFP (10 μM) yield an intracellular concentration of the reporter protein of over 20 μM (51), suggesting that the intracellular concentration of RRM1+2 required to achieve optimal viral inhibition can be attained utilizing dfTat as a delivery agent.

Generate the dfTat:SRSF1-RRMs and Validate its Intracellular Delivery.

Expression clones coding for the chimeric proteins carrying the GB1- and 6His-tags joined to the RRM2, RRM1+2 and EGFP but lacking the Tat CPP are generated. Recombinant proteins are purified in a bacterial system as described above. The optimal concentrations of dfTat and fusion proteins to be utilized in viral inhibition assays are determined by incubating CEM-A cells with varying amounts of recombinant control GB1:EGFP (from 1 to 20 μM) and dfTat (0.5 to 10 μM). The initial range of the dfTat and recombinant GB1:EGFP are determined from the data collected in over 30 primary and stable cell lines. Internalization of the fusion protein is monitored by fluorescence and the results utilized to set up a narrow concentration range for the GB1:RRM2 and GB1:RRM1+2 proteins. Internalization of the chimeric proteins are quantified by confocal microscopy and western blot analysis while cell toxicity is determined as described above. The TatCPP:GB1:RRM12 and TatCPP:GB1:EGFP tagged proteins (described above) are tested in combination with dfTat.

Inhibition of Viral Replication by dfTat/SRSF1:RRMs in CEM-A and Primary CD4+ T Cells.

The inhibition of virion production in CEM-A cells incubated with dfTat and the purified GB1:RRM2, GB1:RRM1+2 or control GB1:EGFP proteins is analyzed. CEM-A cells are infected as described above. The homogenous population of infected cells is then treated with the dfTat/chimeric protein mixture. Viral replication is monitored by infecting the reporter cell line TZM-bl with supernatant collected from the treated and untreated infected CEM-A cells at 24 hours intervals. The therapeutic index for each dfTat/chimeric protein mixture is determined. Next, if the dfTat/GB1:RRM1+2 and dfTat/GB1:RRM2 can inhibit viral replication in primary CD4++T cells, which are isolated, activated than infected as described above, is determined. The infected cells are incubated with the dfTat/GB1:RRM1+2 and dfTat/GB1:RRM2 mixture utilizing the concentration range determined in CEM-A cells. The internalization of the recombinant proteins is monitored by immunohistochemistry and inhibition of viral replication via the reporter cell line TZM-bl while the inhibition of viral transcription and splicing is quantified by qPCR.

The recombinant RRM fusion proteins are expected to be efficiently delivered in T cells via incubation with dfTat. If addition of a NLS to RRM2 increases its antiviral activity, the NLS:RRM2 protein is delivered as a dfTat complex.

As mentioned above, a lentiviral system may be used to deliver an expression cassette coding for the SRSF1 RRM1+2, RRM2 or EGFP to CD4++T cells. Most of the constructs and methods described above can be adapted and utilized to generate and test the antiviral activity of the lentiviral system.

### REFERENCES

1. Paz S, Krainer A R, Caputi M. 2014. HIV-1 transcription is regulated by splicing factor SRSF1. Nucleic Acids Res 42:13812-13823.
2. Paz S, Lu M L, Takata H, Trautmann L, Caputi M. 2015. The SRSF1 RNA Recognition Motifs are strong inhibitors of HIV-1 replication. J Virol doi:10.1128/JVI.00693-15.
3. Jablonski J A, Caputi M. 2009. Role of cellular RNA processing factors in human immunodeficiency virus type 1 mRNA metabolism, replication, and infectivity. J Virol 83:981-992.
4. Mueller N, Berkhout B, Das A T. 2015. HIV-1 splicing is controlled by local RNA structure and binding of splicing regulatory proteins at the major 5′ splice site. J Gen Virol doi:10.1099/vir.0.000122.
5. Caputi M, Freund M, Kammler S, Asang C, Schaal H. 2004. A bidirectional SF2/ASF- and SRp40-dependent splicing enhancer regulates human immunodeficiency virus type 1 rev, env, vpu, and nef gene expression. J Virol 78:6517-6526.
6. Tange T O, Kjems J. 2001. SF2/ASF binds to a splicing enhancer in the third HIV-1 tat exon and stimulates U2A F binding independently of the R S domain. J Mol Biol 312:649-662.
7. Zhu J, Mayeda A, Krainer A R. 2001. Exon identity established through differential antagonism between exonic splicing silencer-bound hnRNP A1 and enhancer-bound S R proteins. Mol Cell 8:1351-1361.
8. Ropers D, Ayadi L, Gattoni R, Jacquenet S, Damier L, Branlant C, Stevenin J. 2004. Differential effects of the S R proteins 9G8, SC35, ASF/SF2, and SRp40 on the utilization of the A1 to A5 splicing sites of HIV-1 RNA. J Biol Chem 279:29963-29973.
9. Jablonski J A, Amelio A L, Giacca M, Caputi M. 2010. The transcriptional transactivator Tat selectively regulates viral splicing. Nucleic Acids Res 38:1249-1260.
10. Amelio A L, Caputi M, Conkright M D. 2009. Bipartite functions of the CREB co-activators selectively direct alternative splicing or transcriptional activation. Embo J 28:2733-2747.
11. Gao G, Guo X, Goff S P. 2002. Inhibition of retroviral RNA production by ZAP, a CCCH-type zinc finger protein. Science 297:1703-1706.
12. Valente S T, Goff S P. 2006. Inhibition of HIV-1 gene expression by a fragment of hnRNP U. Mol Cell 23:597-605.
13. Fong N, Bentley D L. 2001. Capping, splicing, and 3′ processing are independently stimulated by RNA polymerase II: different functions for different segments of the CTD. Genes Dev 15:1783-1795.
14. Pandit R A, Svasti S, Sripichai O, Munkongdee T, Triwitayakorn K, Winichagoon P, Fucharoen S, Peerapittayamongkol C. 2008. Association of SNP in exon 1 of HBS 1L with hemoglobin F level in beta0-thalassemia/hemoglobin E. Int J Hematol 88:357-361.
15. Karn J, Stoltzfus C M. 2012. Transcriptional and posttranscriptional regulation of HIV-1 gene expression. Cold Spring Harb Perspect Med 2:a006916.
16. Bakkour N, Lin Y L, Make S, Ayadi L, Mahuteau-Betzer F, Nguyen C H, Mettling C, Portales P, Grierson D, Chabot B, Jeanteur P, Branlant C, Corbeau P, Tazi J. 2007. Small-molecule inhibition of HIV pre-mRNA splicing as a novel antiretroviral therapy to overcome drug resistance. PLoS Pathog 3:1530-1539.
17. Berkhout B, Silverman R H, Jeang K T. 1989. Tat trans-activates the human immunodeficiency virus through a nascent RNA target. Cell 59:273-282.
18. Gaynor R B. 1995. Regulation of HIV-1 gene expression by the transactivator protein tat.

Curr Top Microbiol Immunol 193:51-77.
19. Fujinaga K, Irwin D, Huang Y, Taube R, Kurosu T, Peterlin B M. 2004. Dynamics of human immunodeficiency virus transcription: P-TEFb phosphorylates R D and dissociates negative effectors from the transactivation response element. Mol Cell Biol 24:787-795.
20. Wada T, Takagi T, Yamaguchi Y, Watanabe D, Handa H. 1998. Evidence that P-TEFb alleviates the negative effect of DSIF on RNA polymerase II-dependent transcription in vitro. Embo J 17:7395-7403.
21. Peterlin B M, Price D H. 2006. Controlling the elongation phase of transcription with P-TEFb. Mol Cell 23:297-305.
22. Bres V, Yoh S M, Jones K A. 2008. The multi-tasking P-TEFb complex. Curr Opin Cell

Biol 20:334-340.
23. Purcell D F J, Martin M A. 1993. Alternative splicing of human immunodeficiency virus type 1 mRNA modulates viral protein expression, replication and infectivity. J Virol 67:6365-6378.
24. Lin S, Fu X D. 2007. S R proteins and related factors in alternative splicing. Adv Exp Med Biol 623:107-122.
25. Zhong X Y, Wang P, Han J, Rosenfeld M G, Fu X D. 2009. S R proteins in vertical integration of gene expression from transcription to RNA processing to translation. Mol Cell 35:1-10.
26. Ji X, Zhou Y, Pandit S, Huang J, Li H, Lin C Y, Xiao R, Burge C B, Fu X D. 2013. S R proteins collaborate with 7S K and promoter-associated nascent RNA to release paused polymerase. Cell 153:855-868.
27. Lin S, Coutinho-Mansfield G, Wang D, Pandit S, Fu X D. 2008. The splicing factor SC35 has an active role in transcriptional elongation. Nat Struct Mol Biol 15:819-826.
28. Long J C, Caceres J F. 2009. The S R protein family of splicing factors: master regulators of gene expression. Biochem J 417:15-27.
29. Fonseca S B, Pereira M P, Kelley S O. 2009. Recent advances in the use of cell-penetrating peptides for medical and biological applications. Adv Drug Deliv Rev 61:953-964.
30. Brooks H, Lebleu B, Vives E. 2005. Tat peptide-mediated cellular delivery: back to basics. Adv Drug Deliv Rev 57:559-577.
31. Madani F, Lindberg S, Langel U, Futaki S, Graslund A. 2011. Mechanisms of cellular uptake of cell-penetrating peptides. J Biophys 2011:414729.
32. Hotchkiss R S, McConnell K W, Bullok K, Davis C G, Chang K C, Schwulst S J, Dunne J C, Dietz G P, Bahr M, McDunn J E, Karl I E, Wagner T H, Cobb J P, Coopersmith C M, Piwnica-Worms D. 2006. TAT-BH4 and TAT-Bcl-xL peptides protect against sepsis-induced lymphocyte apoptosis in vivo. J Immunol 176:5471-5477.
33. Huth J R, Bewley C A, Jackson B M, Hinnebusch A G, Clore G M, Gronenborn A M. 1997. Design of an expression system for detecting folded protein domains and mapping macromolecular interactions by NMR. Protein Sci 6:2359-2364.
34. Clery A, Sinha R, Anczukow O, Corrionero A, Moursy A, Daubner G M, Valcarcel J, Krainer A R, Allain F H. 2013. Isolated pseudo-RNA-recognition motifs of S R proteins can regulate splicing using a noncanonical mode of RNA recognition. Proc Natl Acad Sci USA 110:E2802-2811.
35. Clery A, Jayne S, Benderska N, Dominguez C, Stamm S, Allain F H. 2011. Molecular basis of purine-rich RNA recognition by the human S R-like protein Tra2-beta1. Nat Struct Mol Biol 18:443-450.
36. Vocero-Akbani A, Lissy N A, Dowdy S F. 2000. Transduction of full-length Tat fusion proteins directly into mammalian cells: analysis of T cell receptor activation-induced cell death. Methods Enzymol 322:508-521.
37. Meade A J, Meloni B P, Mastaglia F L, Knuckey N K. 2009. The application of cell penetrating peptides for the delivery of neuroprotective peptides/proteins in experimental cerebral ischaemia studies. J Exp Stroke Transl Med 2:22-40.
38. Liao H X, Sutherland L L, Xia S M, Brock M E, Scearce R M, Vanleeuwen S, Alam S M, McAdams M, Weaver E A, Camacho Z, Ma B J, Li Y, Decker J M, Nabel G J, Montefiori D C, Hahn B H, Korber B T, Gao F, Haynes B F. 2006. A group M consensus envelope glycoprotein induces antibodies that neutralize subsets of subtype B and C HIV-1 primary viruses. Virology 353:268-282.
39. Rousselle C, Clair P, Lefauconnier J M, Kaczorek M, Scherrmann J M, Temsamani J. 2000. New advances in the transport of doxorubicin through the blood-brain barrier by a peptide vector-mediated strategy. Mol Pharmacol 57:679-686.
40. Eum W S, Kim D W, Hwang I K, Yoo K Y, Kang T C, Jang S H, Choi H S, Choi S H, Kim Y H, Kim S Y, Kwon H Y, Kang J H, Kwon O S, Cho S W, Lee K S, Park J, Won M H, Choi S Y. 2004. In vivo protein transduction: biologically active intact pep-1-superoxide dismutase fusion protein efficiently protects against ischemic insult. Free Radic Biol Med 37:1656-1669.
41. Choi J M, Shin J H, Sohn M H, Harding M J, Park J H, Tobiasova Z, Kim D Y, Maher S E, Chae W J, Park S H, Lee C G, Lee S K, Bothwell A L. 2010. Cell-permeable Foxp3 protein alleviates autoimmune disease associated with inflammatory bowel disease and allergic airway inflammation. Proc Natl Acad Sci USA 107:18575-18580.
42. Liou J S, Liu B R, Martin A L, Huang Y W, Chiang H J, Lee H J. 2012. Protein transduction in human cells is enhanced by cell-penetrating peptides fused with an endosomolytic HA2 sequence. Peptides 37:273-284.
43. Snyder E L, Saenz C C, Denicourt C, Meade B R, Cui X S, Kaplan I M, Dowdy S F. 2005. Enhanced targeting and killing of tumor cells expressing the CXC chemokine receptor 4 by transducible anticancer peptides. Cancer Res 65:10646-10650.
44. Vocero-Akbani A M, Heyden N V, Lissy N A, Ratner L, Dowdy S F. 1999. Killing HIV-infected cells by transduction with an HIV protease-activated caspase-3 protein. Nat Med 5:29-33.
45. Misteli T, Caceres J F, Spector D L. 1997. The dynamics of a pre-mRNA splicing factor in living cells. Nature 387:523-527.
46. Caceres J F, Misteli T, Screaton G R, Spector D L, Krainer A R. 1997. Role of the modular domains of S R proteins in subnuclear localization and alternative splicing specificity. J Cell Biol 138:225-238.
47. Cazalla D, Zhu J, Manche L, Huber E, Krainer A R, Caceres J F. 2002. Nuclear export and retention signals in the R S domain of S R proteins. Mol Cell Biol 22:6871-6882.
48. Krebber H, Silver P A. 2000. Directing proteins to nucleus by fusion to nuclear localization signal tags. Methods Enzymol 327:283-296.
49. Liu M T, Hsu T Y, Chen J Y, Yang C S. 1998. Epstein-Barr virus DNase contains two nuclear localization signals, which are different in sensitivity to the hydrophobic regions. Virology 247:62-73.
50. Michael W M, Siomi H, Choi M, Pinol-Roma S, Nakielny S, Liu Q, Dreyfuss G. 1995. Signal sequences that target nuclear import and nuclear export of pre-mRNA-binding proteins. Cold Spring Harb Symp Quant Biol 60:663-668.
51. Lee Y J, Johnson G, Peltier G C, Pellois J P. 2011. A HA2-Fusion tag limits the endosomal release of its protein cargo despite causing endosomal lysis. Biochim Biophys Acta 1810:752-758.
52. Erazo-Oliveras A, Muthukrishnan N, Baker R, Wang T Y, Pellois J P. 2012. Improving the endosomal escape of cell-penetrating peptides and their cargos: strategies and challenges. Pharmaceuticals (Basel) 5:1177-1209.
53. Erazo-Oliveras A, Najjar K, Dayani L, Wang T Y, Johnson G A, Pellois J P. 2014. Protein delivery into live cells by incubation with an endosomolytic agent. Nat Methods 11:861-867.
54. Van Duyne R, Pedati C, Guendel I, Carpio L, Kehn-Hall K, Saifuddin M, Kashanchi F. 2009. The utilization of humanized mouse models for the study of human retroviral infections. Retrovirology 6:76.
55. Caputi M. 2011. The Regulation of HIV-1 mRNA Biogenesis. In Grabowski P (ed), RNA Processing doi:10.5772/20899. InTech.

### Example 3

### Eukaryotic Cells are Efficiently Transduced by SRSF1 Conjugated to a CPP

The goal is to study SRSF1 role in viral latency and reactivation establishing a system to deliver SRSF1 in resting T cells. Approaches currently utilized to transduce leukocytes are based on lentiviral or electroporation systems. Both methodologies have drawbacks such as the lack of homogenous transduction of the cell population and alteration of the cellular gene expression pattern. Both systems are not ideal to deliver expression cassettes in-vivo and, although lentiviral particles are routinely utilized to transduce animals, their use in human subject is heavily restricted. Furthermore, given the overall low transcription level in resting T cells the expression of the transduced genes is often inefficient. To overcome these limitations, CPPs were used to deliver SRSF1 protein to primary T cells. Cell penetrating peptides are a group of short cationic sequences with a remarkable capacity for membrane translocation (Fonseca et al., Advanced drug delivery reviews. 2009; 61(11):953-964) that can be conjugated to large proteins and cross highly selective barriers, such as the intestinal wall or the blood-brain barrier. An HIV-1 Tat-derived, 11 aa long, peptide (hereafter Tat CPP) can deliver a variety of cargoes, from small particles to proteins and nucleic acids (Brooks et al., Advanced drug delivery reviews. 2005; 57(4):559-577; Madani et al., Journal of biophysics. 2011; 2011:414729). Large proteins conjugated to the Tat CPP can be delivered with efficiency in difficult to transfect primary cells, such as lymphocytes (Hotchkiss et al. J Immunol. 2006; 176(9):5471-5477), and in animal models.

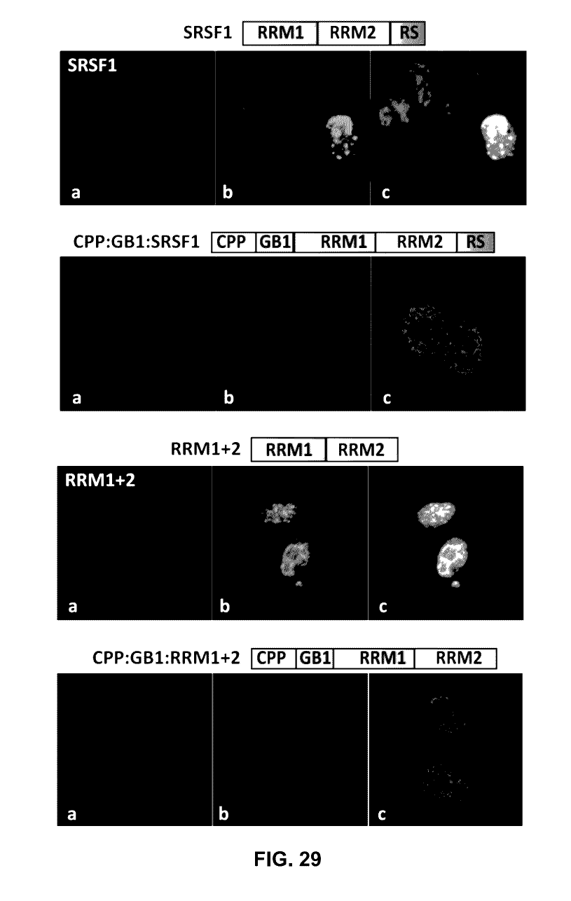

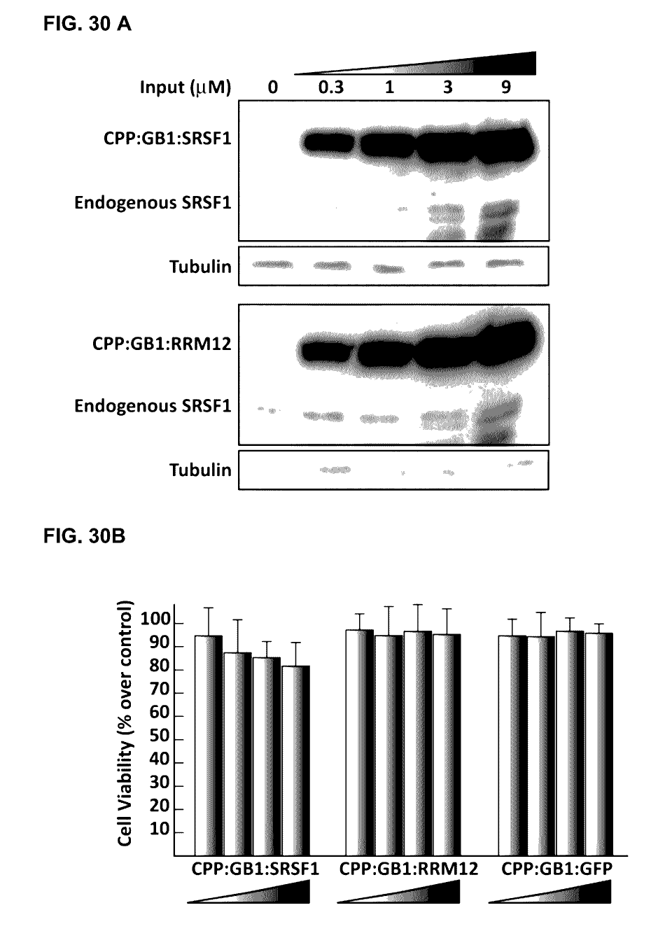

Referring to FIGS. 29 and 30, a bacterial expression system was used to generate a recombinant SRSF1 protein conjugated to the Tat CPP. These data show that the CPP:SRSF1 chimera can transduce stable cell lines (FIG. 29) increasing the concentration of endogenous SRSF1 up to 20 fold (FIG. 30(A)). The biological activity of the recombinant proteins is similar to one of the wild type SRSF1 and exhibits minimal toxicity (FIG. 30(B)). The role of SRSF1 in viral latency is determined by delivering the recombinant CPP:SRSF1 in three latency models of HIV-1 infection.

### Example 4

### Amino Acid Sequences

RRM1+2 (residues 16-195 of SEQ ID NO: 1):
(SEQ ID NO: 17)
CRIYVGNLPPDIRTKDIEDVFYKYGAIRDIDLKNRRGGPPFAFV
EFEDPRDAEDAVYGRDGYDYDGYRLRVEFPRSGRGTGRGGGGGG
GGGAPRGRYGPPSRRSENRVVVSGLPPSGSWQDLKDHMREAGDV
CYADVYRDGTGVVEFVRKEDMTYAVRKLDNTKFRSHEGETAYIR
VKVD
RRM1 (residues 16-91 of SEQ ID NO: 1):
(SEQ ID NO: 18)
CRIYVGNLPPDIRTKDIEDVFYKYGAIRDIDLKNRRGGPPFAFV
EFEDPRDAEDAVYGRDGYDYDGYRLRVEFPRS
RRM2 (residues 121-195 of SEQ ID NO: 1):
(SEQ ID NO: 19)
NRVVVSGLPPSGSWQDLKDHMREAGDVCYADVYRDGTGVVEFVR
KEDMTYAVRKLDNTKFRSHEGETAYIRVKVD

### Other Embodiments

All genes, gene names, and gene products disclosed herein are intended to correspond to homologs from any species for which the composition, kits, and methods disclosed herein are applicable. Thus, the terms include, but are not limited to, genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

Any improvement may be made in part or all of the composition, kits, and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A composition comprising a therapeutically effective amount of SRSF1 protein, a portion of a SRSF1 protein, or a nucleic acid encoding the SRSF1 protein or a portion thereof, for inhibiting replication of a virus in a subject infected with the virus or at risk of being infected with the virus and a pharmaceutically acceptable carrier.

2. The composition of claim 1, wherein the portion of a SRSF1 protein comprises RNA Recognition Motif 1 (RRM-1) and RNA Recognition Motif 2 (RRM-2).

3. The composition of claim 1, wherein the portion of a SRSF1 protein consists of RRM-1 and RRM-2.

4. The composition of claim 1, wherein the portion of a SRSF1 protein comprises RRM-2.

5. The composition of claim 1, wherein the portion of a SRSF1 protein consists of RRM-2.

6. The composition of claim 1, wherein the virus is Human Immunodeficiency Virus type I (HIV-I) and the subject is a human.

7. The composition of claim 1, wherein the SRSF1 protein or the portion of a SRSF1 protein is conjugated to a cell penetrating peptide (CPP).

8. The composition of claim 7, wherein the CPP is Tat-CPP.

9. The composition of claim 7, further comprising an endosomolytic agent.

10. The composition of claim 9, wherein the endosomolytic agent is dfTat.

11. The composition of claim 1, further comprising at least one of: an endosomolytic agent and a CPP.

12. The composition of claim 11, wherein the endosomolytic agent is dfTat.

13. The composition of claim 1, wherein a viral vector comprises the nucleic acid encoding the SRSF1 protein or a portion thereof.

14. The composition of claim 13, wherein the viral vector is selected from the group consisting of: lentiviral vector, retroviral vector, Adeno-Associated Virus vector, Adenovirus vector, and Herpesvirus vector.

15. The composition of claim 1, wherein a cationic liposome comprises the nucleic acid encoding the SRSF1 protein or a portion thereof.

16. A viral vector comprising an expression construct comprising a nucleic acid encoding SRSF1 protein or a portion thereof.

17. The viral vector of claim 16, wherein the nucleic acid encodes a portion of the SRSF1 protein.

18. The viral vector of claim 17, wherein the portion of the SRSF1 protein consists of RRM-1 and RRM-2.

19. The viral vector of claim 16, wherein said viral vector is selected from the group consisting of: lentiviral vector, retroviral vector, Adeno-Associated Virus vector, Adenovirus vector, and Herpesvirus vector.

20. A method for inhibiting viral replication in a subject, comprising administering to the subject a composition comprising SRSF1 protein, a portion of a SRSF1 protein, or a nucleic acid encoding the SRSF1 protein or a portion thereof, in a therapeutically effective amount and a pharmaceutically acceptable carrier.

21. The method of claim 20, wherein the portion of a SRSF1 protein comprises RRM-1 and RRM-2.

22. The method of claim 20, wherein the portion of a SRSF1 protein consists of RRM-1 and RRM-2.

23. The method of claim 20, wherein the portion of a SRSF1 protein comprises RRM-2.

24. The method of claim 20, wherein the portion of a SRSF1 protein consists of RRM-2.

25. The method of claim 20, wherein the virus is HIV-1 and the subject is a human.

26. The method of claim 20, wherein the SRSF1 protein or the portion of a SRSF1 protein is conjugated to a CPP, wherein the CPP is Tat-CPP.

27. The method of claim 20, the composition further comprising at least one of: an endosomolytic agent and a CPP.

28. The method of claim 27, wherein the endosomolytic agent is &Tat.

29. The method of claim 20, wherein a viral vector or a cationic liposome comprises the nucleic acid encoding the SRSF1 protein or a portion thereof.

30. The method of claim 29, wherein the viral vector is selected from the group consisting of: lentiviral vector, retroviral vector, Adeno-Associated Virus vector, Adenovirus vector, and Herpesvirus vector.

31. The method of claim 20, wherein administration of the composition inhibits viral transcription without affecting cell viability in the subject.

32. A method for the prevention, amelioration, or treatment of an HIV-1 infection in a subject, comprising administering to the subject a composition comprising SRSF1 protein, a portion of a SRSF1 protein, or a nucleic acid encoding the SRSF1 protein or a portion thereof, in a therapeutically effective amount and a pharmaceutically acceptable carrier.

33. A kit for inhibiting viral replication in a subject, the kit comprising:
(a) the composition of claim 1;
(b) instructions for use; and
(c) packaging.


SEQUENCE LISTING (19 sequences)

SEQ ID NO: 1 - protein, 248 aa, HOMO SAPIENS
MSGGGVIRGPAGNNDCRIYVGNLPPDIRTKDIEDVFYKYGAIRDIDLKNRRGGPPFAFVEFEDPRDAEDAVYGRDGYDYDGYRLRVEFPRSGRGTGRGGGGGGGGGAPRGRYGPPSRRSENRVVVSGLPPSGSWQDLKDHMREAGDVCYADVYRDGTGVVEFVRKEDMTYAVRKLDNTKFRSHEGETAYIRVKVDGPRSPSYGRSRSRSRSRSRSRSRSNSRSRSYSPRRSRGSPRYSPRHSRSRSRT

SEQ ID NO: 2 - DNA, 5468 bp, HOMO SAPIENS
gttgcgctcccgcgcgtgcgtgttgggatcgaatcgctgtttccttccgcttctcttcctctgtctcccccccatatccgtgcgccgagctgataaaggcgccattttggaggggccgcgggagacgtggtgccgctgcgggctcgctctgccgtgcgctaggcttggtgggaaggcctgttctcgagtccgcgcttttcgtcaccgccatgtcgggaggtggtgtgattcgtggccccgcagggaacaacgattgccgcatctacgtgggtaacttacctccagacatccgaaccaaggacattgaggacgtgttctacaaatacggcgctatccgcgacatcgacctcaagaatcgccgcgggggaccgcccttcgccttcgttgagttcgaggacccgcgagacgcggaagacgcggtgtatggtcgcgacggctatgattacgatgggtaccgtctgcgggtggagtttcctcgaagcggccgtggaacaggccgaggcggcggcgggggtggaggtggcggagctccccgaggtcgctatggccccccatccaggcggtctgaaaacagagtggttgtctctggactgcctccaagtggaagttggcaggatttaaaggatcacatgcgtgaagcaggtgatgtatgttatgctgatgtttaccgagatggcactggtgtcgtggagtttgtacggaaagaagatatgacctatgcagttcgaaaactggataacactaagtttagatctcatgagggagaaactgcctacatccgggttaaagttgatgggcccagaagtccaagttatggaagatctcgatctcgaagccgtagtcgtagcagaagccgtagcagaagcaacagcaggagtcgcagttactccccaaggagaagcagaggatcaccacgctattctccccgtcatagcagatctcgctctcgtacataagatgattggtgacactttttgtagaacccatgttgtatacagttttcctttattcagtacaatcttttcattttttaattcaaactgttttgttcagaatgggctaaagtgttgaattgcattcttgtaatatccccttgctcctaacatctacattcccttcgtgtctttgataaattgtattttaagtgatgtcatagacaggattgtttaaatttagttaactccatactcttcagactgtgatattgtgtaaatgtctatctgccctggtttgtgtgaactgggatgttgggggtgtttgtggttatcttacctggggaagttcttatgtttatcttgcttttcatgtgtctttctgtagacatatctgaagagatggattaagaatgctttggattaaggattgtggagcacatttcaatcattttaggattgtcaaaaggaggattgaggaggatcagatcaataatggaggcaatggtatgactccaagtgctattgtcacagatgaaattggcagtattgaccttatactaaaaggcaggggttaaaaatgattatatacattttccttaaaacacttgcaaacattttattcagttgtctttagctacaattgctttgctttttaaaccttggcaattgtggcaaaattatattgcccattttgtagcaacttattttgctcccttccccccatttttgttttaatagggactaatgtgggaagaactggctaatttgtcacagtgcttagttacaactgttaatgtgtgacctgctgttggtgtacatgtgggtacagggtgtttttaaatccaacaagatagagtataatatcaatactgctaaatctgcatgtcctctgtgtgactgatagagcgttgctatttcatttttttaagacaaaatgaaagcaaaatatagagttccaatgtattggtgtagataatctagttgggaatacttttaagtctcaccttcccctttaaactaatattcataattggttcatatgtttaaaagactttaatttacaaattaaattgcaaatgggagcattagatttagttttagacttaggtgggtagcaatgccagtaaacttaaattacgtaacttcttgcaaccacgaaacctgtaatacgctgtacagtaacaagtgttggcattatcagttgaactgtaaatacaaaatgcttcttccaattagtctctatgatgattaagtttctaaaatttatctgaacaccattcagaaacttgttttggggaatttgatagttattgatgtgcatctgttaaactgatgacagacataactcatcattccccagaaaccttttttgattacagtatctaacattttgcctcctcttttttggttttgctggttataaaggtttggattggagagggctcactggatcccaatccttggagctggatcattggattcaaatcataatgtggataggatagggaggatgaattaccaggattcatggagcgggatcagattaccaggaacataggagtggattcctgccccaaccaaaccgcattcgtgtggatttttttattcaacttaattggctattccaaagattttttttttcctatttttgacgattggagcccttaagatgcacgatggaattgtgttttgcgttttttggtaaaaggagcaaagcgaggacctggagataaacgctggagcaatctccttggaaggattcagcacgagtagatggtaaacatttaaaggggaaagggggggtttgtttaaaatagtaaatcagtaagtcacttctaaatttaaagaaaacaaaattggagttgaagaataagtaggtttccaattggctattgccgttttctttgaaaaaataaacattttttaaaaaactatgcatggttgtcctttttcctcttcatgtaagattctaactgggtctatcagttaatctttaaattgttaagtaagataagattttgactcttgtgttaatgtgttagcaaattaaaagttcttaaaaggcaatctaatggtattagccatcttttattgttaattgtaaaagtcttcaggggaaagcaaaaggggagaataaggcatttgtgtatgtaacttggtaaatgacggtgggggatggatctagcatctgaaagataagcttctctactttgttataaagtggttaaaaaactatagatgctgcttattttctggtggtcatagacaacataggcttttgtgcaaaattggttgatggctactaatgttcacttggagatagcttttgatattctcaatgaaactcatctcaaaaaaaggtaagtattaaatgttaacatcagcacagatgtattagaactgttttttgtttttgagacagagtctcgctctgttctccagactggagggcagtggtgtgatctaggctcactgcaacctccacccctggatttgagtgattctcgtgcctcagtctcccaagtagctgagactacaagtgtgtgccacccttgcccggctaattttgtatttttagtagagatgtggtttctctgtgttacccaggctggtcccaaaactcctggcctcaagtgatctgcctgccttggcctcccaaagtgttaggattacaggtgtgagccaccatgctcagcctgtagaacttttaacccaagtctcatttcttttttgaaagggaagagtgcacaagattaactgcttctttggatgaatcattgttaataaaaagctgggcatttagaattttgccttataagcccttctccaaccataagattattttgtaccaaaaactttggtgttctctaccaaagcagttaaaaacttttagcctgctacttcttgtatttgtctactgacagccccttggtactatttaggttgggggaggggacctaaaataaatagactttaacatttcccttgggtgctaatcatagttggaagttgaatttaaggtgattatttgggtgacaattaaaaacctaaggaaaaccagaaatcttggtagtggaagaaatgtgtaaggtcaccccaatcggtagattttaatgaacgttgtggaatgttgggaagaggggatgttaagttgaatgcagaatttcactaagtacttagtgtaagtttaaggatgtagctctttttatctaagaattcaatgtaatggccaaaaggcagatttactgtttaaaaatttgaataattttacatgacattcttgaaattctaagaagttttatgtgtagaacattttaaaaattcatcagattattaaagggaaaataaatgattaatgataattttggaaggttaatgtgagctagacttaagtaaactttggttcatttgtgttcattgaatgttttggaaatgaccaaaaaatgtaaatggccttcactcaagtttgagtgtttaaagttgaaagatgtgctctactaaaagttatagtaattctaacctcacattgaaatgagacagtattccttgttatacaggctgaatttgaagattagagaggatctaatgtttacttaggtaaaggggcataggttttgtagttaagatgaccagacagctaaaagctgtgatgggaagtatggactgctcctatttatagtctcagaaaatggacctttaggtctctatccgtattggcaattattagaagaaagttacacccttttgaaactacaaaagctgtcttggaatttccccctcttctccctatttatgtccccttagaatattttagggagcctataattattttctaaccaaggaaaaacttaagtctctttaagaagcaattacttttcataacatcagattgaataacccaccttgctgttcagcccacatcctactggaaacaaaaggtaagaaacccattttctggttcttgattgtttgggtctgaattttgtttttaaaactaagctaagtttaatgttttttaaaatgctgtttggaatatgaatagattccccgtaaaatgatttttcctaagttttatgctttagtaaatattcagtgctcacgtctgtgcatcatagtgcttgcgtttaatatgatttattgtagaatctcaacttttcttggtgtttgttgtctttgaaacattgtcttggtcattagggctggtgttttcacatttctgtggtcaaggtggatttcttatgtgtgcctttttgcttactttgtatatgaattttgtaatttaaattgcaagtaagttatatatatgtatttaccataaatagtattaaaagatgagaaactgttagactgaagttctgttgtaacataaccattatttccatcacagtatgaagactgcaaacgcagaaaacagattacagtctcttatccattttttgaaatccaaaaactacgaaaacaaaagattttctgttgttgagctaattaaatgtgaaccctgaccagaaaaaaaaaaaaaaaaaaaaaaaaaaa

SEQ ID NO: 3 - protein, 11 aa, Human immunodeficiency virus type 1
YGRKKRRQRRR

SEQ ID NO: 4 - DNA, 59 bp, Human immunodeficiency virus type 1
gggtctctctggttagaccagatctgagcctgggagctctctggctaactagggaaccc

SEQ ID NO: 5 - DNA, 59 bp, Human immunodeficiency virus type 1
gggtctctctggttagaccagatttgagcctgggagctctctggctaactagggaaccc

SEQ ID NO: 6 - DNA, 59 bp, Human immunodeficiency virus type 1
gggtctctctaggtaggccagatctgagcccgggagctctctggctatatggggaaccc

SEQ ID NO: 7 - DNA, 59 bp, Human immunodeficiency virus type 1
gggtctctcttgttagaccagatacgagcctgggagctctctggctagcaagggaaccc

SEQ ID NO: 8 - DNA, 40 bp, Human immunodeficiency virus type 1
atggcaggaagaagcggagacagcgacgaagagctcatca

SEQ ID NO: 9 - DNA, 40 bp, Human immunodeficiency virus type 1
atggcaggaagaagcggagacagcgacgaagcgctccttc

SEQ ID NO: 10 - DNA, 40 bp, Human immunodeficiency virus type 1
atggcaggaagaagcggagaccgcgacgaagaactcctcc

SEQ ID NO: 11 - DNA, 49 bp, Human immunodeficiency virus type 1
ggacccgacaggcccgaaggaatagaagaagaaggtggagagagagaca

SEQ ID NO: 12 - DNA, 49 bp, Human immunodeficiency virus type 1
ggacccgacaggctcgaaggaatcgaagaagaaggtggagagcaagaca

SEQ ID NO: 13 - DNA, 49 bp, Human immunodeficiency virus type 1
gaacccgacaggcccgaaggaatagaagaagaaggtggagagcgagaca

SEQ ID NO: 14 - protein, 11 aa, Artificial Sequence
  SYNTHETIC CONSTRUCT
LGASWHRPDKG

SEQ ID NO: 15 - protein, 5 aa, Human immunodeficiency virus type 1
GRKKR

SEQ ID NO: 16 - protein, 7 aa, Simian virus 40
PKKKRKV

SEQ ID NO: 17 - protein, 180 aa, HOMO SAPIENS
CRIYVGNLPPDIRTKDIEDVFYKYGAIRDIDLKNRRGGPPFAFVEFEDPRDAEDAVYGRDGYDYDGYRLRVEFPRSGRGTGRGGGGGGGGGAPRGRYGPPSRRSENRVVVSGLPPSGSWQDLKDHMREAGDVCYADVYRDGTGVVEFVRKEDMTYAVRKLDNTKFRSHEGETAYIRVKVD

SEQ ID NO: 18 - protein, 76 aa, HOMO SAPIENS
CRIYVGNLPPDIRTKDIEDVFYKYGAIRDIDLKNRRGGPPFAFVEFEDPRDAEDAVYGRDGYDYDGYRLRVEFPRS

SEQ ID NO: 19 - protein, 75 aa, HOMO SAPIENS
NRVVVSGLPPSGSWQDLKDHMREAGDVCYADVYRDGTGVVEFVRKEDMTYAVRKLDNTKFRSHEGETAYIRVKVD